(19) United States
(12) Patent Application Publication
(10) Pub. No.: US 2025/0169750 A1
(43) Pub. Date: May 29, 2025

(54) A method for assessment of a hemodynamic response to an adenosine receptor agonist stimulation, system for assessment of it and computer readable medium

(71) Applicant: HEMOLENS DIAGNOSTICS SP. Z O.O., Wroclaw (PL)

(72) Inventors: Kryspin MIROTA, Wroclaw (PL); Monika AULICH, Wroclaw (PL)

(21) Appl. No.: 18/839,806

(22) PCT Filed: Feb. 22, 2022
(86) PCT No.: PCT/IB2022/051554

§ 371 Date: Aug. 20, 2024

(51) Int. Cl. A61B 5/00; A61B 3/16; A61B 5/02; A61B 5/021; G16H 50/30

(52) U.S. Cl. CPC A61B 5/4848; A61B 3/16; A61B 5/02028; A61B 5/021; G16H 50/30; A61B 2560/0238

(57) ABSTRACT

A method, a computer-readable medium and a system for assessment of a hemodynamic response to an Adenosine receptor agonist stimulation for a human patient are disclosed. Starting point for the method are non-invasive pressure data in the resting state. Said data are used in pharmacokinetic/pharmacodynamic modeling to arrive at an individual patient-specific response for an adenosine receptor stimulation by an agonist. This response is used to a calibrate lumped-parameter Windkessel type model of a blood circulation. The calibrated model allows for calculating of hemodynamic parameters for the hyperemic state, in particular to calculate flow and pressure values in both coronary arteries inlets. The hemodynamic parameters allow for in silico coronary vessel diagnostic. The effectiveness of the developed method has been confirmed in multi-center clinical trials.

### FIELD OF THE INVENTION

The present invention pertains to method for assessment of a hemodynamic response to an Adenosine receptor agonist stimulation for a human patient, computer-readable medium comprising instructions which, when executed by a computer, cause the computer to carry the steps of mentioned method and system for assessment of a hemodynamic response to an Adenosine receptor agonist stimulation for a human patient. Specifically, the invention pertains to estimation of hemodynamic parameters of coronary circulation of a patient that is in the hyperemic state by modeling an individual patient-specific response for an adenosine receptor stimulation by an agonist. The invention allows for diagnosis and treatment of a coronary insufficiency. In particular, diagnosis and treatment of a coronary heart disease.

### BACKGROUND

According to the National Center for Health Statistics, 5.6% of adults (defined as people having 18 years or more) in the United States had a coronary heart disease (CHD) in 2018. Men tend to have a coronary heart disease more often than women—7.4% vs. 4.1% of adults in the United States. A morbidity risk is increasing with age—15.5% of people aged 65-74 and 23.9% of people 75 and more have a CHD (according to Villarroel M A, Blackwell D L, Jen A (2019) Tables of Summary Health Statistics for U.S. Adults: 2018 National Health Interview Survey, National Center for Health Statistics, Access date: 2021 Dec. 17). A coronary heart disease caused 365 744 deaths in the United States in 2018—almost 13% of all deaths recorded therein in 2018 (see Centers for Disease Control and Prevention, National Center for Health Statistics, National Vital Statistics System: public use data file documentation: mortality multiple cause-of-death micro-data files, Access date: 2021 Dec. 17). An ischemic heart disease caused 623 276 deaths in the European Union in 2017, which accounted for almost 12% of all deaths recorded in 2017 (following Eurostat (2021) Causes of death—deaths by country of residence and occurrence [HLTH_CD_ARO], access date: 2021 Dec. 17).

A diagnostic path of a coronary insufficiency is very complex and multifaceted. With the exception of the initial stage, each of the few following main stages has a gradually increasing reliability which may be connected with increasing invasiveness of the following main stages and with an increasing risk for patients undergoing the diagnostic path (see Scanlon P J et al. (1999) ACC/AHA Guidelines for Coronary Angiography: Executive Summary and Recommendations, Circulation, 99(17), p. 2345-2357, ESC Scientific Document Group (2020) 2019 ESC Guidelines for the diagnosis and management of chronic coronary syndromes, Eur Heart J, 41(3), p. 407-477). A pyramid of a diagnostic accuracy synthetically demonstrates that its culmination and the gold standard is a coronary artery fractional reserve ratio in the form of a fractional flow reserve (FFR) (see Johnson N P et al. (2016) Continuum of Vasodilator Stress from Rest to Contrast Medium to Adenosine Hyperemia for Fractional Flow Reserve Assessment, JACC Cardiovasc Interv, 9(8), p. 757-767). An FFR is described as an elementary ratio of a distal and a proximal pressure, wherein each pressure measurement has to mandatory be made in the hyperemic state. As it is written in an excellent monography of the FFR ratio discoverers Nico Pijls and Bernard De Bruyne: “meaningful measurement of coronary pressure can only be performed in a maximum hyperemic state” (see Pijls N H, Bruyne B D (1997) Coronary Pressure, Springer-Science+Business Media). This quotation does not apply exclusively to an FFR and is also applicable to the accuracy of every CHD diagnostic method. An accuracy of ratios, like FFR or hyperemic ratios, is exceeding the threshold of 95% without any problem. At the same time, a calculated ratio in the resting state is slightly exceeding only 80% and a visual assessment, including the angiography method, is below 70%. As a general principle, a reliable functional assessment is possible only in the hyperemic state.

The hyperemic state in a functional assessment is reached by stimulation of an Adenosine receptor (AR) with a pharmacological agent. There are many commercially available drugs that act as an Adenosine receptor agonist. Adenosine in the form of a medical drug is sold under the trade names: Adenocard, Adenocor, Adenic, Adenoco, Adeno-Jec, Adenoscan etc. An example of a dosage and administration of Adenoscan according to the FDA is: a recommended dose for adults is 140 μg/kg/min administered as a continuous peripheral intravenous infusion for 6 minutes (taken from the U.S. Food & Drug Administration. FDA-Approved Drugs—Labels for NDA 020059, access date: 2021 Dec. 17). Regadenoson is sold under the tradenames of Lexiscan or Rapiscan, Binodenoson under the tradename of CorVue and Apadenoson is sold as Stedivaze. Examples of dosages for those products: 5 ml of Lexiscan (0.4 mg of Regadenoson) injected intravenously within 10 second followed by a radiopharmaceutical and a saline flush (taken from the U.S. Food & Drug Administration. FDA-Approved Drugs—Labels for NDA 022161. Access date: 2021 Dec. 17). According to the DrugBank Database, the III phase of a clinical trial of Apadenoson was terminated and the III phase of a clinical trial of Binodenoson was completed (see DrugBank Online. Access data: 2021 Dec. 17).

Adenosine receptors are integral membrane proteins, which belong to the P1 class of purinergic receptors coupled with the G proteins. G proteins are heterotrimeric protein complexes built from three subunits: α, β and γ. A stable dimeric complex of this protein is built from α and β subunits. Adenosine receptors are divided into four types: A₁, A_{2A}, A_{2B} and A₃. Each receptor is built from seven transmembrane domains. Every receptor has a different number of amino acids and the A_{2A} adenosine receptor is the longest one (see Borea et al. (2018) Pharmacology of Adenosine Receptors: The State of the Art, Physiological Reviews, 98(3), p. 1591-1625, Sheth S et al. (2014) Adenosine receptors: expression, function and regulation, International Journal of Molecular Sciences, 15(2), p. 2024-2052). Every transmembrane domain is built from 21-28 amino acids that form a helix. The C-terminal side of an Adenosine receptor lies within the cytoplasmic area and the N-terminal side lies within the extracellular side of a cell membrane (see Vincenzi M, Bednarska K. Leśnikowski Z J (2018) Comparative Study of Carborane- and Phenyl-Modified Adenosine Derivatives as Ligands for the A2A and A3 Adenosine Receptors Based on a Rigid in Silico Docking and Radioligand Replacement Assay, Molecules (Basel, Switzerland), 23(8), p. 1846).

Adenosine receptors can be found in the nervous, cardiac, pulmonary, digestive, urinary and immune systems (see Borea et al. (2018) Pharmacology of Adenosine Receptors: The State of the Art, Physiological Reviews, 98(3), p. 1591-1625). The activation of each type of adenosine receptors (namely A₁, A_{2A}, A_{2B} and A₃) can affect the functions of a heart. The A₁ adenosine receptor has the highest affinity for Adenosine. The activation of this receptor will slow down the heart rate as well as will cause vasoconstriction and inhibition of the release of a neurotransmitter. The adenosine receptor A_{2A} has the biggest molecular weight of all Adenosine receptors. The biggest number of A_{2A}AR's mRNA can be found in a heart. The activation of the A_{2A}AR leads to an increase in a cAMP level as well as to vasodilatation and inhibition of platelet aggregation. To activate the Adenosine receptor A_{2B}, a high concentration of Adenosine is necessary. There is a low A_{2B} adenosine receptor's expression in a human heart. The activation of the A_{2B}AR leads to increase in a cAMP level as well as to vasodilatation and heart contraction. The biggest number of the A₃ adenosine receptors occurs in the liver, less in the aorta or in a heart. Although, the exact location in a heart is unknown, activation of these receptors has a protective effect on the heart. The activation of the A₃AR also leads to inhibition of the adenylate cyclase (see Shryock J C, Belardinelli L. (1997) Adenosine and adenosine receptors in the cardiovascular system: biochemistry, physiology, and pharmacology. Am J Cardiol. 19; 79(12A):2-10; Wilson C, Mustafa S J (2009) Adenosine Receptors in Health and Disease. Springer Nature; Guieu R et al. (2020) Adenosine and the cardiovascular system: The good and the bad. J. Clin. Med. 9(1366): 1-21).

Most of the Adenosine receptor agonists are purine nucleoside derivatives: Adenosine or Xanthosine. Alkylxanthine derivatives are prototypic antagonists of adenosine receptors. Few parts of Adenosine can potentially interact with receptor's amino acids: five nitrogen atoms of the adenine group (namely, N1, N3, N6, N7 and N9) and three hydroxyl groups of a ribose moiety (namely 2′, 3′ and 5′). There are a lot of different agonists of the Adenosine receptors. A substitution of Adenosine can change the selectivity of the receptors. A substitution with arylalkyl, cycloalkyl and alkyl groups at the N6-position changes selectivity of the A₁ adenosine receptor. A substitution with (thio)ethers, alkynes and secondary amines at the 2-position of Adenosine leads to the formation of new synthetic A_{2A} adenosine selective analogues. A substitution with a selected aryl group at the adenosine's N6-position increases affinity to the A_{2B} adenosine receptor. An increase in selectivity of the A₃ adenosine receptor can be reached via a substitution with a N6-benzyl group or with a substituted benzyl group. Adenosine receptors' antagonists can be obtained in many ways. High affinity and selectivity to the A₁ adenosine receptors can be achieved through modifications of the xanthine core structure at the eight position by an aryl or cycloalkyl. Selectivity to the A_{2A} adenosine receptor can be achieved through the xanthine's eight-position modifications by alkenes. Selectivity to the A_{2B} adenosine receptor can be achieved through modifications of a xanthine core structure at the eight position using aryl groups. A lot of nonxantine classes were defined as the A₃ adenosine receptor antagonists (see Müller C E, Jacobson K A (2011) Recent developments in adenosine receptor ligands and their potential as novel drugs, Biochimica et Biophysica Acta (BBA)—Biomembranes, 1808(5), p. 1290-1308). The Adenosine receptor A_{2A} has an orthosteric binding site. This binding site can be found through the involvement in binding agonists' and antagonists' residues (following Carpenter B, Lebon G (2017) Human Adenosine A2A Receptor: Molecular Mechanism of Ligand Binding and Activation, Frontiers in Pharmacology, 8, p. 898). Caffeine is a natural antagonist of the A_{2A} adenosine receptor. Studies show that Caffeine affects the cardiovascular system, however the results of those studies are inconclusive. A response of the cardiovascular system after Caffeine intake depends on many factors: consumed amount, time of consumption, frequency, liver metabolism and level of absorption. The effect of Caffeine also depends on sex and the differences may be related to steroid hormone concentrations (see Müller C E, Jacobson K A (2011) Recent developments in adenosine receptor ligands and their potential as novel drugs, Biochimica et Biophysica Acta (BBA)—Biomembranes, 1808(5), p. 1290-1308). Based on information from the Protein Data Bank, Caffeine binds to the A_{2A} adenosine receptor using atoms O13 and N7 and a hydrogen bond and using atom N3 and a Cation-Pi interaction. Regadenoson is a selective A_{2A} adenosine receptor agonist. Regadenoson is metabolized in plasma slower than Adenosine. A dose-dependent increase in a coronary blood flow can be caused by Regadenoson (according to Müller C E, Jacobson K A (2011) Recent developments in adenosine receptor ligands and their potential as novel drugs, Biochimica et Biophysica Acta (BBA)—Biomembranes, 1808(5), p. 1290-1308).

Further selective A_{2A} adenosine receptors agonists are Binodenoson (BND) and Apadenoson (APA); Apadenoson is more selective than Binodenoson. In this comparison, Regadenoson is the least selective. The affinity of APA and BND to an Adenosine receptor is equal. The average onset of a binding action is between 1 and 2 minutes. Apadenoson has longer duration of the action than Bidodenoson: 10-20 minutes. The Binodenoson's time of duration is less than 5 minutes. A dose of APA and BND is calculated depending on patient's weight. Both adenosine agonists are introduced through an intravenous bolus (see Elkholy K G et al (2021) Regadenoson Stress Testing: A Comprehensive Review with a Focused Update, Cureus, 13(1), p. e12940).

Adenosine receptors take part in regulation of a vascular tone, however this is not fully understood. It is proven that a A_{2A} Adenosine receptor activation regulates a vascular tone, but also that the A_{2B} Adenosine receptors are involved in small artery relaxation. Adenosine receptors interact with each other to regulate the coronary vessels. Regulation of a coronary microcirculation mediated by Adenosine can be either endothelium-dependent or endothelium-independent. In a basal tone control mediated by the A2A Adenosine receptor, a reactive hyperemia nitric oxide is involved. It is also a part of a A_{2A}AR activation mediated by Adenosine and of a A_{2A}AR-K_{ATP} line in reactive hyperemia (see Zhang Y et al. (2021) Adenosine and adenosine receptor-mediated action in coronary microcirculation, Basic Research in Cardiology, 12(116)).

Due to the adenosine receptors' importance and role, pharmacokinetics and pharmacodynamics of its endogenous and exogenous agonists and antagonists are subjects of research. The most studied and most surveyed to date is Adenosine. Kern et al. were testing Adenosine as an agent for the determination of a coronary vasodilator reserve. Adenosine was injected intravenously in doses of 50, 100 and 150 μg/kg/min for 3 minutes during tests. 34 patients participated in these tests: 17 with and 17 without a coronary artery disease (CAD) (see Kern M J et al. (1991) Intravenous adenosine: continuous infusion and low dose bolus administration for determination of coronary vasodilator reserve in patients with and without coronary artery disease, Journal of the American College of Cardiology, 18(3), p. 718-29). Alexopoulos et al. performed tests to investigate the effects of increasing Adenosine doses on P_{d}/Pₐ. Thirty-eight patients were treated with a standard adenosine dose (140 μg/kg/min) introduced through the femoral vein with a repeat P_{d}/Pₐ assessment. Eight patients refused to participate in tests with a high Adenosine dose. Thirty patients were treated with a high adenosine dose (200 μg/kg/min) with a repeat P_{d}/Pₐ assessment. The patients had a stable coronary artery disease or exhibit an acute coronary syndrome under certain conditions. A mean systolic blood pressure decreased more after the high Adenosine dose of 200 μg/kg/min in comparison to the standard Adenosine dose of 140 μg/kg/min. A mean diastolic blood pressure decreased to lower values after the high Adenosine dose in comparison to treatment using the standard Adenosine dose. A heart rate (HR) increased more after injection of the high Adenosine dose (see Alexopoulos D et al. (2016) Effect of High (200 μg/kg per Minute) Adenosine Dose Infusion on Fractional Flow Reserve Variability, Journal of the American Heart Association, 5). The Adenosine effect on hemodynamics was measured using a cardiovascular magnetic resonance imaging in a test performed by Thomas et al. Adenosine was administrated intravenously into 25 healthy patients in a dose of 140 μg/kg/min for 6 minutes. The test results showed a small decrease in a mean systolic blood pressure following an Adenosine injection, but no changes in a mean diastolic blood pressure following the Adenosine injection and a high increase in a heart rate following the Adenosine injection (see Thomas D et al. (2017) Effects of adenosine and regadenoson on hemodynamics measured using cardiovascular magnetic resonance imaging, Journal of Cardiovascular Magnetic Resonance, 19). Another test where researchers were studying infused Adenosine in a normal dose for 6 minutes and its influence on hemodynamics was performed by Mishra et al. Three hundred forty-eight healthy volunteers participated in this test. The obtained results showed that a mean systolic blood pressure and a mean diastolic blood pressure changes were very small. Only a heart rate increased significantly (following Mishra R et al. (2005) Quantitative relation between hemodynamic changes during intravenous adenosine infusion and the magnitude of coronary hyperemia—Implications for myocardial perfusion imaging, Journal of the American College of Cardiology, 45, p. 553-8). Lee et al. were testing the use of Adenosine as an alternative method to testing by exercises (known as a pharmacologic stress testing) on 15 healthy volunteers. Adenosine was intravenously infused in a dose of 140 μg/kg/min for 6 minutes. In line with the previous tests, a mean systolic blood pressure decrease was small. A mean diastolic blood pressure decreased by a small value and a heart rate increased significantly (for more details see Lee J H et al. (1994) Biokinetics of thallium-201 in normal subjects: comparison between adenosine, dipyridamole, dobutamine and exercise, Journal of nuclear medicine: official publication, Society of Nuclear Medicine, p. 535-41). The experimental results showed that it is not possible to mathematically model the effect of an Adenosine receptor stimulation based on a simple relationship, which is necessary to estimate the effect of adenosine on heart hemodynamics. In every conducted test, the heart rate increased, but by a different value. At the same time, the differences in the HR in groups with a coronary artery disease (CAD) and without a CAD weren't significant. Results of a systolic blood pressure were ambiguous. There were differences between the values of a systolic blood pressure at the baseline and during a peak hyperemia following an Adenosine infusion in each test. These results showed that after an Adenosine infusion, a systolic blood pressure decreased, but in an experiment performed by Brown et al. (see Brown L et al. (2021) A comparison of standard and high dose adenosine protocols in routine vasodilator stress cardiovascular magnetic resonance: dosage affects hyperemic myocardial blood flow in patients with severe left ventricular systolic impairment, Journal of Cardiovascular Magnetic Resonance, 23) a systolic blood pressure did not change after an Adenosine infusion (in heart failure (HF) with left ventricular ejection fraction (LVEF)≥40% group of 16 patients—rest 124±16, stress 124±11) or increased (in HF LVEF <40% group of 20 patients—rest 119±16, stress 122±18). Test results described a decrease of a diastolic blood pressure or presented no changes of diastolic blood pressure values. Vasu et al. were testing Adenosine, Regadenoson and Dipyridamole on fifteen healthy volunteers. The relative potency of these vasodilators was evaluated using quantified stress and rest myocardial perfusion. The results were obtained using a cardiovascular magnetic resonance (CMR). Stress tests were performed within a few days following 400 μg bolus of Regadenoson, 0.56 mg/kg of Dipyridamole and 140 μg/kg/min of Adenosine. Each test started with recording of a rest perfusion imaging. After 20 minutes, a vasodilation stress imaging was recorded during the peak, i.e., 3-4 minute following an Adenosine infusion, 4 minutes after a Dipyridamole infusion and 70 seconds following a Regadenoson infusion. A myocardial blood flow (MBF) and a myocardial perfusion reserve (MPR) were quantified by a fully quantitative model constrained deconvolution. A higher values of stress MBF and of a heart rate (HR) were recorded following a Regadenoson injection in comparison to the ones recorded for Dipyridamole or Adenosine. After adjusting of values of the stress MBF to the values of HR, there were no differences between Adenosine and Regadenoson, but the differences between Dipyridamole and Regadenoson were still observed. An unadjusted MPR was higher for Regadenoson than for Adenosine and Dipyridamole. After adjusting the values of a stress MPR to the values of HR, there were no differences between Regadenoson and Adenosine, but the differences between Regadenoson and Dipyridamole and also between Adenosine and Dipyridamole were still observed (see Vasu S et al. (2013) Regadenoson and adenosine are equivalent vasodilators and are superior than dipyridamole—a study of first pass quantitative perfusion cardiovascular magnetic resonance, Journal of cardiovascular magnetic resonance: official journal of the Society for Cardiovascular Magnetic Resonance, 15, p. 85). Averaged values from different clinical trials of systolic pressure (SYS), diastolic pressure (DIA) and heart rate (HR), as well as coronary artery disease (CAD), can be found in the cited references.

Non-invasive diagnostic methods of a circulatory failure based on computational fluid dynamics (CFD) technology are trying slowly, though with some struggles, to gain the trust of the medical environment. The current results of a validation study do not provide an unambiguous recommendations (following Nakanishi R, Budoff M J (2016) Noninvasive FFR derived from coronary CT angiography in the management of coronary artery disease: technology and clinical update, Vase Health Risk Manag, 12, p. 269-78, Kruk M et al. (2016) Workstation-based calculation of CTA-based FFR for intermediate stenosis, JACC Cardiovasc Imagin, 9(6), p. 690-9, Chung J H et al. (2017) Diagnostic performance of a novel method for fractional flow reserve computed from noninvasive computed tomography angiography (NOVEL-FLOW Study), Am J Cardiol, 120(3), p. 362-368, Wang Z Q et al. (2019) Diagnostic accuracy of a deep learning approach to calculate FFR from coronary CT angiography, J Geriatr Cardiol, 16(1), p. 42-48, Fujimoto S et al. (2019) Diagnostic performance of on-site computed CT-fractional flow reserve based on fluid structure interactions: comparison with invasive fractional flow reserve and instantaneous wave-free ratio, Eur Heart J Cardiovasc Imaging, 20(3), p. 343-352, Tang C X et al. (2020) CT FFR for ischemia-specific cad with a new computational fluid dynamics algorithm: A Chinese multicenter study, JACC Cardiovasc Imaging, 13(4), p. 980-990, Nozaki Y O et al. (2021) Comparison of diagnostic performance in on-site based CT-derived fractional flow reserve measurements, Int J Cardiol Heart Vasc, 35, p. 1-5).

Even if only limited to a single in silico evaluation platform, satisfactory values of sensitivity understood as a true positive rate (TPN) and specificity understood as a true negative rate (TNR) can be reached for a single clinical trial (TPR/TNR=93.2%/82.9% followed by 74%/67% and 85%/79%). Any results obtained during similar trials can become questionable for a so-called “grey zone”. This can be better understood when using an example. For instance, few years ago a team from the Imperial College London proved in a review paper that “For vessels with FFR-CT values below 0.60, 0.60 to 0.70, 0.70 to 0.80, 0.80 to 0.90, and above 0.90, diagnostic accuracy of FFR-CT was 86.4% (95% CI, 78.0%-94.0%), 74.7% (95% CI, 71.9%-77.5%), 46.1% (95% CI, 42.9%-49.3%), 87.3% (95% CI, 85.1%-89.5%), and 97.9% (95% CI, 97.9%-98.8%), respectively” (taken from Cook C M et al. (2017) Diagnostic Accuracy of Computed Tomography-Derived Fractional Flow Reserve: A Systematic Review, JAMA Cardiol, 2(7), p. 803-810). This review paper produced controversy and polemics. It may be concluded that the authors' assessment was not very precise since, above all, an accuracy of 50% is a diagnostic equivalent of a flip of coin. This level of accuracy is not considered to be satisfactory.

It may be presumed that this ambiguous view of the in silico technology is caused by its relatively early stage of development. Most developments in the in silico technology are still in the research phase and more time is needed to transform them into commercially-developed products. Certainly, there are still many elements of these developments that will require improvements. The most promising approach is the one concerning an invasive coronary artery fractional flow reserve (FFR), since an FFR is currently described as a gold standard test. Nico Pijls and Bernard De Bruyne, the already-mentioned authors of an FFR index concept, wrote in their research summary monograph that there “ . . . are three reasons why, in the past, coronary pressure measurement has not been useful for assessment of flow: First: the first prerequisite for reliable intracoronary pressure recording is to use ultrathin pressure-monitoring guide wires, Second: pressure measurements are only meaningful at maximum coronary hyperemia, and Third: it is not the gradient, but the remaining distal coronary pressure which determines myocardial perfusion.” (taken from Pijls N H J, de Bruyne B (2000) Coronary Pressure, Springer-Science+Business Media, p. 52). Most likely in the upcoming years we will have a possibility to observe development of recommendations for improvement for in silico technologies. Today, the first recommendation is pertaining to a measuring instrument which for computing technologies should be interpreted as an improvement of the resolution of medical imaging and discrete geometry models. This recommendation does not raise significant doubts and is well established. Further, the second recommendation pertains to improvement towards describing very significant physiological circumstances. In particular, those which can make any measurement more or less reliable and, as a consequence, be responsible for not fully showing the coronary insufficiency state. The third recommendation appears to be expected and also does not raise any objections. This recommendation pertains to what should be assessed or measured as a diagnostic index. Just like in an invasive procedure, in in silico, a fractional flow reserve ratio is calculated by relating to a distal and a proximal pressure. Peculiarly, said proximal pressure, while obligatory for every clinician, is treated differently by everyone dealing with in silico technologies.

The presented above in-depth prior art review shows that there is a need for a reliable in silico method of assessing the condition of the coronary system for a human patient. The invention disclosed and claimed herein addresses this need.

### BRIEF DESCRIPTION OF THE INVENTION

The forthcoming description is to better understand the principle and benefits of the invention claimed in the appended claims. Therefore, it is not meant to be limiting in any sense.

The present invention approaches the problem of assessment of the coronary system with the use of a proper description of the hyperemic state. The inventors concluded that a reliable functional assessment of the coronary system can be made only if it is known how the coronary system will behave in the hyperemic state. Since the hyperemic states is induced by the stimulation of Adenosine receptors using agonists of said receptors, an assessment of hemodynamic state of a patient following exposure to Adenosine receptors agonists is needed for said reliable functional assessment. This invention models a patient's response to an Adenosine receptor agonist stimulation.

In one aspect, the invention relates to a method for assessment a hemodynamic response to a stimulation with an Adenosine receptor agonist for a human patient. The method can be realized as a computer-implemented invention.

The method according to the invention involves estimation of a response of one or more Adenosine receptors to stimulation with one or more agonists of these receptors. This estimation comprises of arriving at parameters of a systolic and a diastolic pressure as well as a heart rate at the hyperemic state using a pharmacokinetic/pharmacodynamic (PK/PD) model. This is done in a step called mapping.

Mapping of resting hemodynamic parameters describing the human patient in the resting state to hyperemic hemodynamic parameters describing the human patient in the hyperemic state using said pharmacokinetic/pharmacodynamic model requires that the pharmacokinetic/pharmacodynamic model is a non-linear model. The pharmacokinetic/pharmacodynamic model has to also include Michaelis-Menten, Hill-Langmuir, Black-Leff, multi-pathway and/or multi-species blood-tissue exchange model (for more details, see for example Bassingthwaighte J B, Wang C Y, Chan I S (1989) Blood-tissue exchange via transport and transformation by capillary endothelial cells, Circ Res, 65(4):997-1020; Kroll K, Deussen A, Sweet I R (1992) Comprehensive model of transport and metabolism of adenosine and S-adenosylhomocysteine in the guinea pig heart. Circ Res, 71(3):590-604). The use of at least one model, e.g., of Hill-Langmuir, is sufficient for the invention. The use of combinations of models can provide in some cases better results.

The resting hemodynamic parameters include a patient's systolic pressure, a patient's diastolic pressure and/or a patient's heart rate. In one embodiment, the resting hemodynamic parameters consists only of a patient's systolic pressure, a patient's diastolic pressure and a patient's heart rate. For example, if the resting hemodynamic parameters consist only of a patient's systolic pressure in the resting state, a patient's diastolic pressure in the resting state and a patient's heart rate in the resting state, the hyperemic hemodynamic parameters consist only of a patient's systolic pressure in the hyperemic state, a patient's diastolic pressure in the hyperemic state and a patient's heart rate in the hyperemic state.

These resting parameters can be measured directly from a patient in any non-invasive way or taken from an earlier non-invasive measurement made for that patient. Alternatively, the resting hemodynamic parameters can be assumed using a non-invasively recorded continuous patient's pressure waveform. The waveform can be recorded at the same time as the resting hemodynamic parameters or not. For a reliable assumption of the resting hemodynamic parameters the waveform has to be recorded for a time window that includes at least one entire cycle of a heart of the patient. More said time windows will provide improved results. In order to be able to assume said resting hemodynamic parameters, the waveform has to be representative for the resting state and cannot be a waveform recorded for the hyperemic state. In a different embodiment, the resting hemodynamic parameters further includes a non-invasively recorded continuous resting patient's pressure waveform.

In an embodiment, the PK/PD model is also calculating a patient's pressure waveform in the hyperemic state using said non-invasively recorded continuous resting patient's pressure waveform. This is not beneficial in terms of pace of calculations but it is beneficial as the patient's pressure waveform in the hyperemic state can be used during a parametric identification step of the method.

The method according to the invention subsequently involves obtaining a model that describes the patient in the hyperemic state. This is achieved by performing a parametric identification of a lumped-parameter model using hyperemic hemodynamic parameters describing the human patient in the hyperemic state. In this step, empirical parameters of the lumped-parameter model are adjusted using hyperemic hemodynamic parameters describing the human patient in the hyperemic state. Thereby the empirical parameters of the lumped-parameter model and the whole model describes the patient in the hyperemic state. In embodiments, the empirical parameters can include resistance, compliance, inertance, parameters of the time-varying elastance concept and/or parameters of the myocardial fiber-strain/stress concept. In other embodiments, the empirical parameters further can include parameters of a myocardium vessel interaction, parameters of a blood circulatory system (BCS) component, a heart chamber pressure-volume (HPV) component and/or a coronary blood flow (CBF) component. The empirical parameters can include any other additional parameters which are used to adjust the model to the patient and/or which are beneficial in terms of stability of the model.

The lumped-parameter model has to be of a Windkessel type which means that said model has to be built using variations of blocks which were developed by Windkessel. The lumped-parameter model has to describe at least in part hemodynamics of the patient which should be understood as involving description of a relevant part of the blood circulatory system or the whole blood circulatory system of the human patient. The level of description of hemodynamics is tuned to what information is needed to be provided by the invention.

The lumped-parameter model that is representative for the hyperemic state obtained using the method according to the invention allows for in silico diagnosis of the patient. In particular, it allows for calculating of flow parameters which may include a pressure, a flow rate and/or cardiac time intervals that include, but are not limited to, a heart cycle duration and an ejection time. The mentioned flow parameters are hyperemic transients and they can be used to obtain a reliable diagnosis of a human patient. In particular, said transients can be used to diagnose and treat a coronary heart disease. In silico diagnosis is beneficial as it allows for by-passing of all drawbacks of undergoing invasive procedures on the patient.

The method may include gathering or use of demographic and health data which affect circulation hemodynamic in patients' bodies. Said data may specifically cover data that affect pulse propagation in patients' bodies. The demographic and health data may include patient's gender, age, body height, general fitness assessment and/or current medication. The current medication includes, but is not limited to, a beta-adrenergic blocking agent, an angiotensin-converting-enzyme inhibitor and/or an antiarrhythmic agent. The current medication can include all medications that may affect a human heart. The data may be of any patient, including data taken from a database. The data is used to get a first approximation of values of the empirical parameters of the lumped-parameter model. This shortens the time needed to arrive at the results of the method. It may also be beneficial in terms of stability of calculations. In one embodiment, this is done by find relationships between empirical parameters and the data. The first approximation allows for faster calculations and the method arrives at the lumped-parameter model describing the human patient in the hyperemic state faster and stable. In another embodiment, when the data is gathered or taken from patients that suffer from a coronary heart disease, the method arrives at the lumped-parameter model describing the human patient in the hyperemic state even faster and even more stable. The reason for this is that the empirical parameters of the model are much closer to the ones that describe the patient in the hyperemic state for which a diagnosis is needed.

The method according to the invention may also comprise recording in a non-invasive manner a patient's pressure waveform. This waveform has to represent the patient's resting state. The patient's pressure waveform is recorded for a chosen time window that includes at least one cycle of a patient's heart. The patient's pressure waveform can be recorded in a continues manner. Preferably, for the radial artery. The preferred technique for recording of the patient's pressure waveform is finger-cuff photoplethysmography and/or applanation tonometry. Non-invasive recoding means registration that does not involve any type of surgery and/or significant health risks; in some embodiments, said registration does not include insertion of any probe into a patient's body.

A crucial development provided by the invention is a reliable approach to modelling of a myocardium vessel interaction (MVI) that forms part of a three-component model of the invention. This interaction is need for an improved calculation of a flow that is present in the coronary artery. The three-component model of the invention offers reliable description of hemodynamics in the hyperemic state. This covers also description of hemodynamics of patients with complex medical conditions. The three-component model of the invention offers adaptability and good computational performance.

In another aspect, the invention pertains to a computer-readable [storage] medium comprising instructions which, when executed by a computer, cause the computer to carry the steps of a method according to the embodiments herein disclosed.

In another aspect, the invention pertains to a system for assessment of a hemodynamic response to an Adenosine receptor agonist stimulation for a human patient, wherein the system comprises a measuring means for non-invasively measuring resting hemodynamic parameters of the human patient, wherein the resting hemodynamic parameters include a patient's systolic pressure, a patient's diastolic pressure and/or a patient's heart rate; and a computer system adapted to perform the steps of a method defined in any embodiment of the invention. This aspect allows for simultaneous measurements and diagnosis. This is beneficial in terms of arriving at diagnosis faster for a patient. Alternatively, this allows for separation of a place of diagnosis where a patient is with a place where the calculations are done. This is beneficial in many ways as, for example, some calculations may require more computer power which cannot be provide in the place of diagnosis. Another benefit is a possibility to use less computational powerful devices to make measurements (e.g., handheld devices) in comparison to a setup in which the same device is used for calculations and measurements/recordings.

In one embodiment, the system for assessment of a hemodynamic response to an Adenosine receptor agonist stimulation for a human patient as defined in this disclosure includes the computer system as defined in this disclosure that further comprises a registering means for non-invasive continuous registration of a resting pressure waveform for the human patient. This aspect allows for even broader simultaneous measurements/recordings and diagnosis. This is even more beneficial with respect to the embodiment described above.

In one embodiment, the system for assessment of a hemodynamic response to an Adenosine receptor agonist stimulation for a human patient as defined in this disclosure, wherein the computer system as defined in this disclosure comprises at least one computer adapted to perform mapping of resting hemodynamic parameters describing the human patient in the resting state to hyperemic hemodynamic parameters describing the human patient in the hyperemic state using a pharmacokinetic/pharmacodynamic model, and at least one computer adapted to perform a parametric identification of a lumped-parameter model using the hyperemic hemodynamic parameters describing the human patient in the hyperemic state to arrive at the lumped-parameter model that is describing the human patient in the hyperemic state, and wherein said at least one computer adapted to perform mapping of resting hemodynamic parameters describing the human patient in the resting state to hyperemic hemodynamic parameters describing the human patient in the hyperemic state using a pharmacokinetic/pharmacodynamic model is configured to communicate directly or indirectly with said at least one computer adapted to perform a parametric identification of a lumped-parameter model using the hyperemic hemodynamic parameters describing the human patient in the hyperemic state to arrive at the lumped-parameter model that is describing the human patient in the hyperemic state. This system of the invention allows for separation of places where various calculations are done. This allows for more flexibility and adaptability to the situation that is in a place where there is a patient to be diagnosed. There are situations in which this may benefit in faster diagnosis.

Further characteristics and advantages of the invention will be more apparent from the detailed description of non-limiting embodiments and from the appended figures. It should be understood that embodiments presented in the description and in the claims, unless stated otherwise, can be combined together in any order and number to produce new embodiments that form part of the disclosure. The description comprises a great number of references to prior art documents, in particular to scientific journals. The full disclosure of said references is part of this disclosure.

### BRIEF DESCRIPTION OF FIGURES

The invention will now be described in more detail with reference to the appended figures in which:

FIG. 1 presents an embodiment of the invention implementing also optional steps,

FIG. 2 presents a block diagram of generalized form of a three-component model (A), and three basic building functional blocks for a lumped-parameter model that can be used to construct a blood circulatory system (B), a heart chamber pressure-volume (C) and a coronary blood flow (D),

FIG. 3 presents a block diagram that illustrates in detail one of embodiments of the present invention of a three-component model for use in determining hemodynamics,

FIG. 4 presents results of a PK/PD approximation of transition from the resting to the hyperemic state in a patient with a typical response obtained with an embodiment of the present invention,

FIG. 5 presents results of a PK/PD approximation of transition from the resting to the hyperemic state in a patient with a humped response obtained with an embodiment of the present invention,

FIG. 6 presents results of a regressive model used for the selected internal empirical parameters of a three-component model describing a patient in the hyperemic state for an embodiment of the present invention, and

FIG. 7 presents a comparison of values of pressures measured in vivo in the hyperemic states with values of pressures calculated in silico using the three-component model according to an embodiment of the present invention.

### DETAILED DESCRIPTION

The present invention aims at diagnosing coronary insufficiency and delivers developments in specifying model and methodology of describing hyperemia in silico. As already described, achieving the hyperemic state involves using stimulation of a specific pharmacological adenosine receptor (generally, the ADORA2 receptor is the target) in clinical practice. The stimulation is done using Adenosine, Regadenoson, Binodenoson or Apadenoson administered by an intracoronary bolus (i.e.) or an intravenous infusion (i.v.). An agonist binding effect is in the form of an Adenosine receptor signaling pathway cascade which ultimately leads to a coronary arteries vasodilation. This results in reducing to minimum a coronary vascular resistance (CVR) and in increasing to the achievable maximum a coronary blood flow. The invention approaches this with the division into two aspects: arriving at a coronary flow as such and tracking changes taking place during the hyperemic state.

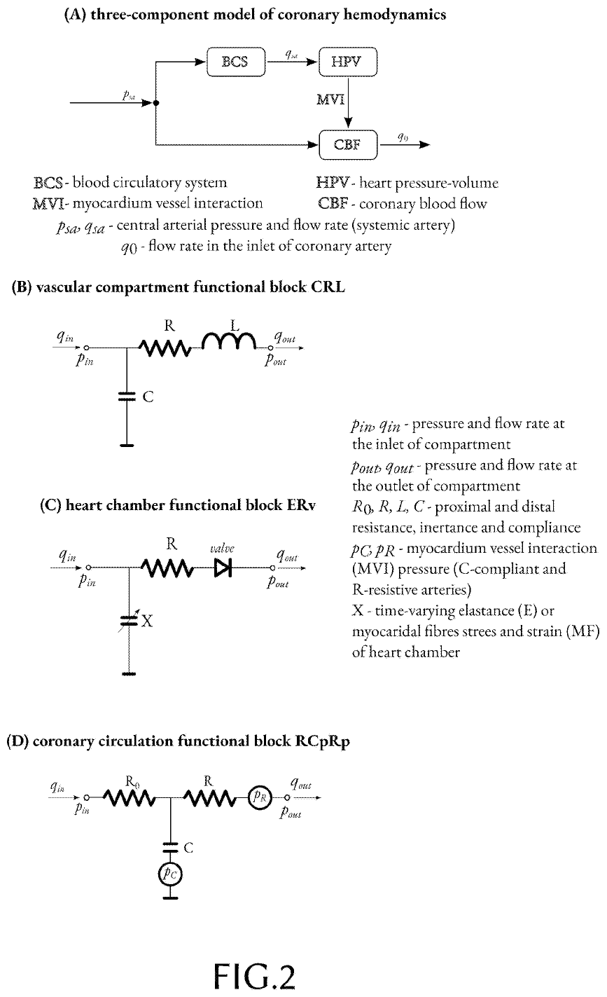

In an embodiment of the present invention, a hemodynamic model is defined by a three-component model (3CM) of coronary hemodynamics (see FIG. 2 A). Said model includes the following coupled components: a blood circulatory system (BCS) component, a heart chamber pressure-volume (HPV) component and a coronary blood flow (CBF) component. Each of the components is created using one or more functional block, wherein the functional block must be specific for a given component (for BCS, HPV and CBF we use CRL, ERv and RCpRp, respectively. CRL, ERv and RCpRp are depicted on FIG. 2 B, FIG. 2 C and FIG. 2 D, respectively). A ERv functional block comprises a valve which is a heart valve modeling diode. The diode should be understood as a model of a one-direction flow, in line with the terminology used in this field.

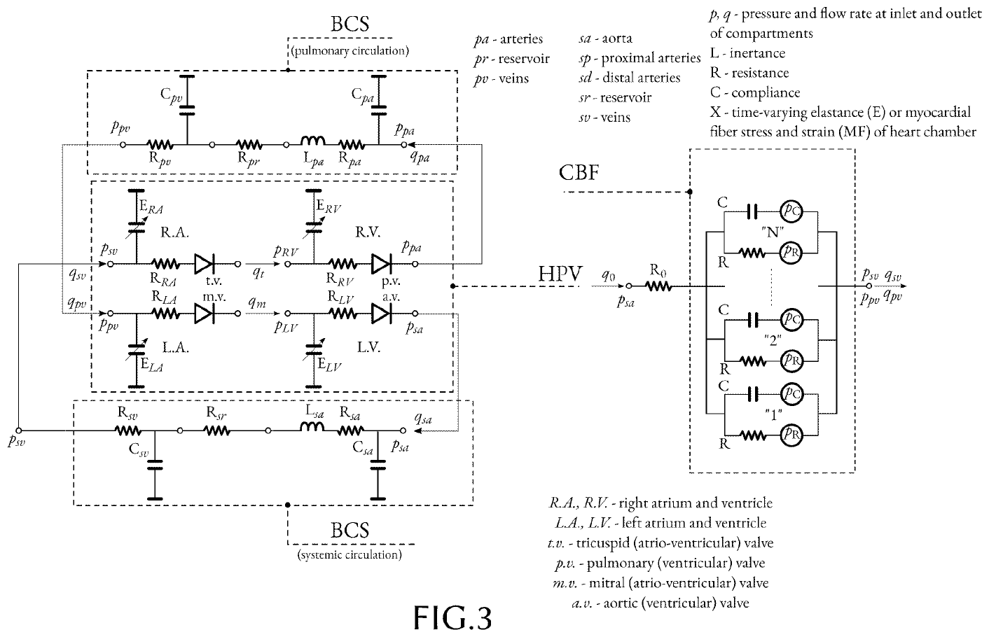

The forthcoming description will provide detailed relations in the form of equations which can be used for each said block. It should be noted that, for example, the parameter of pressure (p) is one equation may or may not correspond to the of pressure (p) in another equation. Which pressure has to be used in each equation can be derived, for example, from FIG. 3. The used form of equations is justified since every block can be used multiple times in a given component. Listening of all possible configurations with respective relations would unnecessarily expand the description and due to its complexity possibly be damaging for the understanding of the invention.

In case of a CRL functional block, a flow parameters' relation between input and output can be presented using the following elementary equations:

$$\{ \begin{matrix}{q}_{in} = C \frac{{dp}_{in}}{dt} + {q}_{out} \\ {p}_{in} = {Rq}_{out} + L \frac{{dq}_{out}}{dt} + {p}_{out}\end{matrix} . \qquad ( 1 )$$

where C, R and L are compliance, resistance and inertance, respectively.

In an embodiment, a systemic (left heart circuit) and pulmonary (right heart circuit) circulation model has a form of at least two CRL functional blocks serially connected for each circuit (so n-compartment n-CRL, where n=2, 3, 4, . . . ). This is beneficial in terms of arriving at a model that offers a reliable description while not requiring too high computational power to provide results. Going further, a heart model is formed by 2-chamber type blocks describing the atrium and ventricle (FIG. 2 C), separately for the left and the right blood circulation side. Heart chamber hemodynamics can be described by a system of differential equations given below:

$$\{ \begin{matrix}{q}_{in} = \frac{dV}{dp} \frac{dp}{dt} + {q}_{out} \\ {q}_{out} = \frac{〈 {p}_{in} - {p}_{out} 〉}{R}\end{matrix} , \qquad ( 2 )$$

where symbol < > represents Macaulay brackets. For closing this system of equations, it is preferable and sometimes necessary to know the changes in blood pressure (p) in all four heart chambers with cyclic changes in volume (V) during systole and diastole. Unlike within a vascular tree, where compliance is assumed to satisfy C(t)≠const, in the invention changes are significant and do not have a passive character. In a preferred embodiment of the invention, the heart chamber vulnerability is described by the time-varying elastance concept (E) as a reciprocal of a time-varying compliance (see Suga H (1969) Time course of left ventricular pressure-volume relationship under various enddiastolic volume, Jpn Heart J, 10(6), p. 509-15) which has the following form:

$$E ( t ) = \frac{1}{C ( t )} = \frac{p ( t )}{V ( t ) - {V}_{0}} , \qquad ( 3 )$$

where V₀ is the unloaded volume defined as an end-systolic heart chamber p=f(V) relationship.

The practical implementation of relationship (3) requires approximation for the E(t) compliant with the use of heart physiology. In a preferred embodiment of the invention, the approximation is done with a periodic double-Hill function (see Stergiopulos N et al. (1996) Determinants of stroke volume and systolic and diastolic aortic pressure, Am J Physiol, 270(6 Pt 2), p. H2050-9) with the following equation:

$$\frac{E - {E}_{min}}{{E}_{max} - {E}_{min}} = {E}_{n} = A \cdot f ( {t}_{n} , {a}_{1} , {n}_{1} ) \cdot ( 1 - f ( {t}_{n} , {a}_{2} , {n}_{2} ) ) , \qquad ( 4 a )$$

while

$$f ( {t}_{n} , a , n ) = \frac{{t}_{n}^{n}}{{a}^{n} + {t}_{n}^{n}} , \qquad ( 4 b )$$

where: E has a value defined by the equation (3), Eₘₐₓ, Eₘₐₓ—minima and maxima of the equation (3), A—a dimensionless scale factor, n—a coefficient of sigmoidicity. and tₙ=(t % T)/tₘₐₓ—a normalized value of time (%—modulo operator, T—heart period, tₘₐₓ—the moment of time for which the elastance function reaches its maximum value).

A proper description of circulation requires a model to be complemented with a p-V relation describing myocardium. In one embodiment, said relation is expressed using a time-varying elastance (E), in another—by implementing the myocardial fiber-strain/stress concept (see Mirota K (2008) Constitutive Models of Vascular Tissue, Solid State Phenomena, 144, p. 100-105, Avazmohammadi R et al. (2019) A Contemporary Look at Biomechanical Models of Myocardium, Annual Review of Biomedical Engineering, 21, p. 417-442, Voigt J U, Cvijic M (2019) 2- and 3-Dimensional Myocardial Strain in Cardiac Health and Disease, JACC Cardiovasc Imaging, 12(9), p. 1849-1863). The fiber-strain/stress concept assumes that for all thin-walled and near-rotationally-symmetric geometries, the fiber stress (s_{f}) changes are proportional to a bulk modulus 1/V·dp/dV˜s_{f}, and therefore—after integration—a relation for the cavity pressure (p) and fiber stress (σ_{f}) ratio is expressed by:

$$\frac{p}{{σ}_{f}} = \frac{1}{3} ln ( 1 + \frac{{V}_{w}}{V} ) . \qquad ( 5 )$$

The myocardial fiber-strain/stress concept (MF) is much more sophisticated when compared to the time-varying elastance concept (E). The reason is that it covers deformation and strain in a myocardial muscle fiber. On the other hand, the time-varying elastance concept (E) is beneficial in terms of pace of calculations.

The last component of the three-component model (depicted on FIG. 2 A) for use in determining coronary hemodynamics is the coronary blood flow (CBF) component. This component comprises a RCpRp functional block showed on FIG. 2 D. Relations between its input and output can be presented by a general equation given below:

$${q}_{in} = C \frac{d}{dt} ( {p}_{in} - {R}_{0} {q}_{in} - {p}_{C} ) + \frac{〈 {p}_{in} - {R}_{0} {q}_{in} - {p}_{R} - {p}_{zf} 〉}{R} + {q}_{out} , \qquad ( 6 )$$

where p_{zf} is zero flow pressure in coronary flow.

In a preferred embodiment of present invention, a zero flow pressure p_{zf} is assumed to be equal 14.8±7 mmHg for patients with a stable angina pectoris, 22.5±9.1 mmHg for patients with a non-Q-wave myocardial infraction, and 37.1±1.9 mmHg for patients with Q-wave myocardial infraction (see Nanto S et al. (1996) Zero flow pressure in human coronary circulation, Angiology, 47(2), p. 115-22, Van Herck P L et al. (2007) Coronary microvascular dysfunction after myocardial infarction: increased coronary zero flow pressure both in the infarcted and in the remote myocardium is mainly related to left ventricular filling pressure, Heart, 93(10), p. 1231-7). Assumption is beneficial for the pace of calculations and for shortening the time needed for providing diagnosis.

A myocardium vessel interaction (MVI) is used for describing characteristics of the coronary circulation. A flow in the coronary artery is a function of arterial intel and outlet conditions and a function of MVI. To arrive at more reliable values of a coronary flow an MVI is needed and at the same time there is no known in the art unambiguous and reliable modelling strategy of the MVI. Development of modelling strategy of the MVI is a very important component of a system developed by the inventors of the present invention. The pressure p_{C} as well as p_{R} describes MVI effects, which are fundamental for said modelling (with reference to Boileau E et al. (2015) One-dimensional modelling of the coronary circulation. Application to noninvasive quantification of fractional flow reserve (FFR), Computational and Experimental Biomedical Sciences: Methods and Applications, 21, p. 137-155, Bruinsma T et al. (1988) Model of the coronary circulation based on pressure dependence of coronary resistance and compliance, Basic Res Cardiol, 83, p. 510-524, Epstein S et al. (2015) Reducing the number of parameters in 1D arterial blood flow modeling: less is more for—patient-specific simulations, American Journal of Physiology, Heart and Circulatory Physiology, 309(1), p. H222-H234, Mohrman D et al. (2013) Cardiovascular physiology, McGraw-Hill, Lange N Y et al. (2013) Cardiovascular physiology, Elsevier, Zamir M (2005) The physics of coronary blood flow, Springer-Verlag). If we focus only on passive effects, which are inherent in the hyperemic state, the values of pressure are controlled by two mechanisms: (i) interstitial, cavity-induced extracellular pressure CEP=μ₁p_{V}(t), and (ii) shortening-induced intracellular pressure SIP=μ₂Eₙ(t) (as described in Algranati D et al. (2010) Mechanisms of myocardium-coronary vessel interaction, American Journal of Physiology. Heart and Circulatory Physiology, 298(3), p. 11861-11873, Mynard J P et al. (2014) Scalability and in vivo validation of a multiscale numerical model of the left coronary circulation, American Journal of Physiology. Heart and Circulatory Physiology, 306(4), p. H517-11528, Westerhof N et al. (2006) Cross-talk between cardiac muscle and coronary vasculature, Physiological Reviews, 86(4), p. 1263-1308). Therefore, in a preferred embodiment of the invention, a coronary flow throttling pressure is calculated as follows:

$${p}_{R , C} = {k}_{R , C} ( CEP + SIP ) = {k}_{R , C} ( {μ}_{1} p ( t ) + {μ}_{2} {E}_{n} ( t ) ) , \qquad ( 7 )$$

where: p—interstitial, intracavity pressure, k_{R,C}—resistive and compliant part of a coronary tree coefficient, CEP—cavity-induced extracellular pressure, SIP—shortening-induced intracellular pressure, μ₁—cavity-induced extracellular pressure coefficient, μ₂—shortening-induced intracellular pressure, and Eₙ—normal elastance of equation 4a. This embodiment offers a reliable description needed for a reliable diagnosis while at the same time has a balanced computational requirements.

By using functional blocks listed above: CRL, ERv and RCpRp (as presented on FIG. 2 B, FIG. 2 C, FIG. 2 D, respectively) a complete hemodynamic model can be built. An overview of the complete hemodynamic model is given in FIG. 2 A, while a more detailed structure of this model according to a preferred embodiment of the invention is presented on FIG. 3. For the model to become useful in a method, which is a practical implementation thereof, the model has to be completed by a set of empirical parameters. In the most direct approach to achieve this goal one can reach out to numerous publications. Last decades of researching a blood flow modeling resulted not only in thousands of articles in scientific journals but also in many monographs on this subject (for example: Rideout V C (1991) Mathematical and computer modeling of physiological systems, Prentice Hall, Spaan J A E (1991) Coronary blood flow mechanics, distribution, and control, Springer Science+Business Media Dordrecht, Hoppensteadt F C, Peskin Ch (1992) Modeling and Simulation in Medicine and the Life Sciences, Springer-Verlag; Ottesen J T, Olufsen M S, Larsen J K (2004) Applied Mathematical Models in Human Physiology, SIAM, Zamir M (2005) The Physics of Coronary Blood Flow, Springer Science+Business Media, van Meurs W L (2011) Modeling and Simulation in Biomedical Engineering: Applications in Cardiorespiratory Physiology, Willem van Meurs, McGraw Hill).

Insofar as this approach successfully solve the explicit method formulation problem, it gives infinitesimal possibilities of considering patient-specific variability.

In an embodiment of the invention, one can determine or measure selected (preferably those which are easy to measure) anatomic or hemodynamic parameters for every analyzed case individually and then calibrate empirical parameters of a model using said anatomic or hemodynamic parameters (see Kirk J A et al. (2013) A priori identifiability analysis of cardiovascular models, Cardiovasc Eng Technol, 4(4), p. 500-512, Stergiopulos N et al. (1996) Determinants of stroke volume and systolic and diastolic aortic pressure, Am J Physiol, 270(6 Pt 2): p. H2050-9). In a preferred embodiment of the present invention it is a continuous non-invasive measurement using a photoplethysmogram.

The problem solved by the invention is approached differently in the state of the art. Below there is a brief introduction into the strategies not used in the invention and forming the state of the art. This is relevant for a proper understanding of the invention.

In the state of the art, there is a strategy based on a statistical approach. In the simplest attempt it can be tried to estimate the patient-specific empirical parameters variability by using population-based statistical rules suitable to demographical or general medical data (following Burattini R, Di Salvia P O (2007) Development of systemic arterial mechanical properties from infancy to adulthood interpreted by four-element windkessel models, J Appl Physiol (1985), 103(1), p. 66-79, McVeigh G E et al. (1999) Age-related abnormalities in arterial compliance identified by pressure pulse contour analysis: aging and arterial compliance, Hypertension, 33(6), p. 1392-8, Kassab G S (2019) Coronary Circulation, Anatomy, Mechanical Properties, and Biomechanics, Springer Science+Business Media, Westerhof B E et al. (2020) Pressure and Flow Relations in the Systemic Arterial Tree Throughout Development From Newborn to Adult, Front Pediatr, 8, p. 251).

A hemodynamic parameter that is very easy to measure is pressure at the main artery level. At the same time pressure is a direct reference data for model prediction results. In this regard available is the auscultatory method that uses a stethoscope and a sphygmomanometer or the oscillometric method.

Next, cardiac output estimates are implemented based on an echocardiographic assessment (attempts in this regard can be found in Lang R M et al. (2015) Recommendations for cardiac chamber quantification by echocardiography in adults: an update from the American Society of Echocardiography and the European Association of Cardiovascular Imaging, J Am Soc Echocardiogr, 28(1), p. 1-39, Dumesnil J G et al. (1995) A new, simple and accurate method for determining ejection fraction by Doppler echocardiography, Can J Cardiol, 11(11), p. 1007-14) or using a computed tomography assessment heart anatomy.

Based on the knowledge of the mass or volume of the heart chambers, it is estimated according to myocardial blood flow (MBF) [ml/min 100 g] (see Anderson H V et al. (2000) Coronary artery flow velocity is related to lumen area and regional left ventricular mass, Circulation, 102(1), p. 48-54) which is expressed by:

$$MBF = 8 \cdot ( 7 \cdot {10}^{- 4} HR \cdot SYS - 0.4 ) , \qquad ( 8 )$$

or directly by estimating myocardial oxygen consumption MVO₂ [ml O₂/min 100 g] (very extensive review of potentially available estimates is provided by Baller D et al. (1979) Validity of myocardial oxygen consumption parameters, Clin Cardiol, 2(5), p. 317-27). The strategy of selecting empirical parameters of the circulation model, such as a preferred embodiment of FIG. 3, which comprises selection (calibration) of the empirical parameters of the model according to the available partial characteristics of hemodynamics (such as pressure, myocardial blood flow, etc.) was identified as being used. This methodology allows to directly relate to real and actual hemodynamics state, but with significant limitations.

Unfortunately, in the context of using the strategy described above to coronary heart disease diagnostic functional assessment, the strategy presented above is deficient. Its disadvantage is accessibility of some parameters for target hemodynamics state—if a model prediction would relate to the hyperemic state, its parameters have to be established for the hyperemic and not for the resting state. In fact, it is possible to measure a pressure (SYS, DIA) and a heart rate (HR) in the resting state, or even continuously using a pressure waveform without difficulty or complications. However, it is unreasonable to administer adenosine or another vasodilatants used in pharmacologic stress testing during said measurements of SYS, DIA and HR.

The prior art offers various other strategies to approach the problem of the invention. In the simplest approach it can be tried to recreate a hyperemic effect by assuming some arbitrary change of the main hemodynamic parameters. For example, it can be assumed that a coronary artery resistance decreases by a constant factor of four, while an aortic blood pressure decreases approximately by 10%, and a heart rate increases approximately by 10% (as disclosed in Taylor C A (2012) Method and system for patient-specific modeling of blood flow. U.S. Pat. No. 8,157,742B2; Taylor C A (2021) Method and system for image processing to determine blood flow. U.S. Patent No. 20210282860A1). As it was shown by an extensive review of clinical trials results, there is a very high risk that results obtained with those prior art approaches will be far from reality. Mainly since a patient-specific variability here is huge and what is missing here is a direct correlation with demographical or general medical data.

Observations of a high patient-specific variability for an adenosine receptors stimulation were made well before development of any models to quantify said variability. As a consequence, while looking for suggestions for explaining mechanisms of this phenomenon, it is worth to mention earlier prior art approaches. This will help to better understand the details of the invention.

To test dose-response kinetics of Adenosine injected intravenously and intracoronary, a test on 39 patients was performed by Wilson et al. At the beginning, a coronarography was performed to diagnose a chest pain syndrome in patients. Thirty-nine patients were divided into two groups wherein eight patients had microcirculatory abnormalities associated with a ventricular hypertrophy of an uncertain etiology, 31 had atypical chest pain due to angina pectoris, mildly or normal stenotic epicardial coronary arteries, a normal coronary flow reserve (CFR) and a normal left ventricular function. Researchers were testing kinetic responses to doses of Adenosine administered by an intravenous infusion, an intracoronary infusion and an intracoronary bolus. A single dose of the intracoronary bolus was between 2 and 16 μg, the intracoronary infusion was in the range of 10-240 μg/min and the intravenous infusion was in the range of 35-140 μg/kg/min. A Doppler coronary catheter 3F was used to measure a coronary blood flow velocity (CBFV). The biggest change in a CBFV was calculated as the ratio of a peak CBFV after an Adenosine or an Papaverine intracoronary bolus to a rest CBFV (ΔCBFV). An index of the change of the total coronary resistance was calculated as below.

$$Δ TCRI = ( \frac{peak hyperemic coronary blood flow velocity}{mean arterial pressure at peak hyperemia} ) / ( \frac{CBFV at rest}{arterial pressure at rest} ) \qquad ( 9 a )$$

where changes in a CBFV during the intracoronary infusion of Adenosine were calculated as

$$Δ CBFV = \frac{mean {CBFV}_{110 - 120 after infusion}}{basal CBFV}$$

while changes in a CBFV during the intravenous infusion of Adenosine were calculated as below

$$Δ CBFV = \frac{peak mean CBFV during infusion}{basal blood flow velocity} \qquad ( 9 b )$$

A peak coronary vasodilation was performed safely by an intracoronary Adenosine and almost a peak hyperemia could be reached after an intravenously injected of 140 μg/kg/min of Adenosine (see Wilson R F et al. (1990) Effects of adenosine on human coronary arterial circulation, Circulation, 82(5), p. 1595-1606). Another important observation was that different doses of Adenosine were having a different impact on the test results.

Researchers led by Sparv were investigating influence of increasing doses of Adenosine on a fractional flow reserve (FFR), a mean arterial pressure (MAP), a heart rate (HR) and on discomfort (which was measured by Visual-Analogue-Scale (VAS)). Eighty-seven patients participated in the tests (10 patients got an atrioventricular block after an Adenosine injection and were excluded from the tests, and two resigned due to a huge discomfort). During the evaluation of an FFR, patients were getting a standard dose of intravenous Adenosine of 140 μg/kg/min and then a high dose of intravenous Adenosine of 220 μg/kg/min. Another group of patients was also analyzed, but this group included Caffeine that was consumed less than 6 hours before an FFR by forty-three patients. The results showed no significant changes in the FFR results, MAP and HR between the standard and the high Adenosine dose. The only differences between the standard and the high dose of Adenosine were observed as a patients' higher discomfort after the high Adenosine dose. During the tests, MAP and HR were monitored and recorded as the baseline with intervals of 30 seconds. A statistical analysis was performed using the Wilcoxon matched-pairs signed rank test and a linear regression model correlation of Adenosine doses. A Bland-Altman plot was created to show an agreement and, for continuous hemodynamic variables of MAP and HR, an area under a curve (AUC) was analyzed (see Sparv D et al. (2017) Assessment of increasing intravenous adenosine dose in fractional flow reserve, BMC Cardiovascular Disorders, 17, p. 1-9).

Another prior approach was developed by Robert Wilson and his group. Robert Wilson et al. from a department of the Medicine of University of Minnesota provided not only a multilateral Adenosine kinetics assessment, but also a total coronary resistance index TCRI (see eq. (9a)). It is indeed parallel to the linear resistance law appearing in many branches of physics (for example as Ohm or Hagen-Poiseuille law). It can be assumed that his work was an attempt of a deeper insight into this problem, but it did not bring anything close to a breakthrough. Nevertheless, it is inspiring that this work showed a slightly different strategy towards contemporary hemodynamics circulatory model empirical parameters selection for the hyperemic state. Namely, instead of dubious ad hoc assumptions, as the flow or heart rate may change in a specific case, one can try to make a hypothetical structural relationship between them (see Sharma P et al. (2012) A framework for personalization of coronary flow computations during rest and hyperemia, Annu Int Conf IEEE Eng Med Biol Soc, p. 6665-8; Sharma et al. (2019) Non-invasive functional assessment of coronary artery stenosis including simulation of hyperemia by changing resting microvascular resistance. U.S. patent Ser. No. 10/373,700B2; Itu L M et al. (2020) Framework for personalization of coronary flow computations during rest and hyperemia. U.S. patent Ser. No. 10/622,110B2).

By defining a coronary circulation resistance by analogy to a systemic circulation resistance (normally magnitude of 9 . . . 20 mmHg min/l) with a:

$$R = \frac{MAP}{Q} , \qquad ( 10 a )$$

and the TRCI index as quotient of a definite resistance—here for the resting and the hyperemic state expressed as:

$$TRCI = \frac{{R}_{hyper}}{{R}_{rest}} = {( \frac{MAP}{Q} )}_{hyper} \cdot {( \frac{Q}{MAP} )}_{rest} . \qquad ( 10 b )$$

This equation allows for obtaining a resistance parameter for the hyperemic state based on a resistance parameter in the resting state. This is because Rₕᵢₚₑᵣ=TRCI·Rᵣₑₛₜ. It is clear that the effectiveness of this strategy will depend on determination of a TRCI index value.

TRCI can be also determined such that it will include a correction with respect to HR. See equation (11) (as indicated by Sharma P et al., 2012 which was based on a clinical trial result of McGinn A L. et al. (1990) Interstudy variability of coronary flow reserve. Influence of heart rate, arterial pressure, and ventricular preload, Circulation, 81(4), p. 1319-30) which has the following form:

$$TRCI = \{ \begin{matrix}0.0016 \cdot HR + 0.1 , & HR \leq 100 bpm \\ 0.001 \cdot HR + 0.16 , & HR > 100 bpm\end{matrix} \qquad ( 11 )$$

The strategy based on the TRCI index concept, in some cases, can provide flexibility or even can be effective in terms of recreation of an influence of a patient-specific variability. However, this strategy can be effectively applied only when the set of the empirical parameters in the model is small. In the example cited above (specifically, Sharma P et al. (2012)), the recalculation related to the transition from the resting state to the hyperemia relates only to resistances. In other words, this strategy will work only for a selected model and it will fail for any models that are designed to describe more complex medical cases. One cannot omit the individual medical parameters of a patient.

Various embodiments of the present invention utilize a different strategy when compared to the previously described, in particular different to the use of population-based statistical data or TRCI. The invention strategy is based on mapping between the resting and the hyperemic state which is done by chosen hemodynamic parameters (such as SYS, DIA etc.) which are fitted to the result provided by the pharmacokinetic/pharmacodynamic (PK/PD)-model of Adenosine metabolism.

Important approaches to the PK/PD-model of Adenosine metabolism will be described in the forthcoming paragraphs. In the area of the PK/PD-model of Adenosine metabolism, a cardinal point is a classic work of a team from the Istituto di Clinica Medica, Università di Siena. Blardi et al. performed tests, where an Adenosine kinetic analysis was carried out one minute after an Adenosine infusion in doses of 2.5, 5 or 10 mg (38, 79 and 48 μg/kg, respectively) and after an infusion of 200 μg/kg administered over a period of 10 min which was followed by a dose of 400 μg/kg administered over a period of 10 min. Twenty two healthy volunteers were divided into 4 groups: group A (6 volunteers, a 2.5 mg intravenous Adenosine administered by a 1 minute infusion), group B (6 volunteers, a 5 mg intravenous Adenosine administered by a 1 minute infusion), group C (5 volunteers, a 10 mg intravenous Adenosine administered by a 1 minute infusion), group D (5 volunteers, a 200 μg/kg intravenous Adenosine infusion administered over a period of 10 min and followed by a 400 μg/kg intravenous Adenosine infusion administered over a period of 10 min). The researchers were collecting blood samples at the time of an adenosine infusion (time 0) and at selected points in time: 1, 3, 5, 7, 10 and 15 minutes in groups A, B and C, and at time 0, and 5, 10, 15, 20, 21, 25 and 30 minutes in group D. One minute after an Adenosine infusion, adenosine concentration increased, a mean clearance decreased and a half-life increased. After 20 minutes, an Adenosine plasma concentration reached a peak compared to the concentration reached following a 5 mg Adenosine infusion administered over a period of 1 min, but a mean clearance and a half-life were different. The Adenosine concentration returned to the baseline after 5-15 minutes flowing the stop of infusion. The Adenosine plasma concentration was examined by the HPLC. A pharmacokinetic analysis between the adenosine concentration after a certain period in time and the baseline concentration was conducted. The Adenosine concentration C(t) in groups A, B and C was calculated as

$$C ( t ) = A [ \frac{{e}^{β} - 1}{β} ] {e}^{- β t} \qquad ( 12 )$$

where: t—time, A and βμparameters estimated by a non-linear regression (see Blardi P et al. (1993) Pharmacokinetics of exogenous adenosine in man after infusion, European journal of clinical pharmacology, 44(5), p. 505-507).

Even though Blardi's work differs from contemporary PK/PD models used in the field, it is from the early 90s of the 20th century and it is a very important reference point.

Another very interesting prior art attempt at quantification kinetics of Adenosine was the work of Davis et al. Researchers were trying to define effects of Glibenclamide (that blocks K_{ATP} channels) and N^{ω}-Nitro-L-arginine methyl ester (L-NAME) (that blocks a nitric oxide synthesis). Adenosine was administrated via a coronary infusion (using doses of 10-1000 μg/min). Dogs were at first properly prepared and then anesthetized using mechanical ventilation. Left tracheotomy was made and catheters were placed into the descending aorta and the left atrium. A Konigsberg micromanometer was placed in a left ventricle through a left ventricular apex. A Transonic Systems transit time ultrasound flow probe and a hydraulic occluder were placed in the proximal portion of the left circumflex coronary artery or in the left anterior descending coronary artery. An angiocatheter 22-gauge connected to a small-bore tubing was placed in the artery, distal to the flow probe. Onto the left atrial appendage bipolar pacing leads were sewn. A value of Adenosine plasma concentration that is needed to reach 50% of that maximal conductivity increase (ED₅₀) in the basal condition increased three times after administering of 1 mg/kg or 10 mg/kg of L-NAME. ED₅₀ increased dose-dependently after a Glibenclamide administration. K_{ATP} channels and NO synthesis blockade were additive with ED₅₀ 15-times increase. Glibenclamide and L-NAME caused an increase in systemic pressure and a reduction in coronary conductance. During the tests, hemodynamic was monitored in an analog form at a sampling rate for 30 s. For each individual, variables mean values were recorded every 30 s. An Adenosine plasma concentration was calculated with the use of the Olsson approach (see equation 14). Dose-response adenosine data were fitted to a sigmoidal model expressed below as:

$$f ( x ) = \frac{{e}^{A {x}^{2}}}{B + C {e}^{A {x}^{2}}} + D \qquad ( 13 )$$

where: x—log adenosine plasma concentration; f(x)—conductance; A, B, C, D—constants (see Davis C A et al. (1998) Coronary vascular responsiveness to adenosine is impaired additively by blockade of nitric oxide synthesis and a sulfonylurea, Journal of the American College of Cardiology, 31(4), p. 816-822). In this review there is a direct reference to a typical sigmoidal Adenosine receptors response model.

Further prior work was done by Tune et al. They were analyzing the role of Adenosine in coronary exercise hyperemia on ten dogs. Catheters were placed in the aorta and the coronary sinus. A flow probe was placed on the circumflex coronary artery. An Adenosine concentration in a cardiac interstitial was estimated from a coronary and an arterial venous plasma concentration using mathematical models. A coronary blood flow (CBF), a consumed oxygen by the myocardium, a heart rate (HR) and an aortic pressure were measured at rest and during exercise on a treadmill with and without an Adenosine receptor blockade. During the exercise, oxygen consumption increased 4.2-times, CBF 3.8-times, HR 2.5-times and mean blood pressure (MBP) remained unchanged for the dogs from a control group. An Adenosine plasma concentration in coronary veins changed very little during the exercise. An estimated Adenosine interstitial concentration was well below the threshold for a coronary vasodilation. The oxygen consumption or a CBF, both during rest and the exercise, were almost stable following an Adenosine receptor blockade. An Adenosine concentration in coronary veins and the estimated interstitial adenosine concentration did not change to compensate the receptor blockade. The Adenosine arterial plasma concentration [ADO] was calculated using the following equation:

$${[ A D O ]}_{art . plasma} = \frac{ADO infusion rate}{Blood flow \cdot ( 1 - Hct )} \cdot {[ ADO ]}_{infusate} \qquad ( 14 )$$

where: [Ado]—adenosine concentration, Hct—hematocrit. Subsequently, the Hill equation dose-response adenosine data were fitted using the following equation:

$$q = {q}_{min} + \frac{( {q}_{max} - {q}_{min} ) \cdot {[ ADO ]}^{H}}{{[ ADO ]}^{H} + E {D}_{50}^{H}} \qquad ( 15 )$$

where: q—coronary blood blow, qₘᵢₙ—measured rest coronary blood flow, qₘₐₓ—measured peak coronary blood flow, [A]—calculated adenosine concentration in artery, ED₅₀—effective dose in 50%, H—Hill ratio (see Tune J D et al. (2000) Adenosine is not responsible for local metabolic control of coronary blood flow in dogs during exercise, American Journal of Physiology-Heart and Circulatory Physiology, 278(1), p. H74-H84).

The invention approaches PK/PD models differently and this will be presented below. For achieving a biochemical control, the nature developed a very precise adjustment mechanism in the form of an enzyme catalyzed reaction. Apart from substrates S and products P, biocatalyst E emerges in the setup, whose activity is subtly regulated, appropriate for the needs and circumstances. This relation can be summarized in the simplest form as a chemical equation: E+S↔ES→E+P. It is reasonable to assume that an enzyme-substrate complex (ES) concentration during the reaction does not change significantly. Similarly, it can be assumed that the equilibrium occurs between the concertation of the complex and the substrates: k₁[E][S]−k₋₁[ES]=k₂[ES]. By combining all this, we get a simple relationship describing the rate of a product formation depends on the substrate accessibility:

$$\frac{d P}{d t} = \frac{{v}_{max} [ S ]}{{K}_{M} + [ S ]} \qquad ( 16 a )$$

The above-presented equation was discovered by Leonor Michaelis and Maud Menten during their work on fermentation kinetics. Over time, this equation became very useful for describing broadly understood enzymatically catalyzed processes (see Rodwell V W et al. (2018) Harpers Illustrated Biochemistry, McGraw-Hill Medical). In the case of complex biochemical systems, the number of reactants can be large and the reactions often form a cascade system. A good example are Purine receptors (and Adenosine receptors, in particular) for which the reactions usually run in parallel with the use of n catalytic centers and thus (following Mirota K (2013) Hemodynamic aspects of atherogenesis) we arrive at the following equation:

$$\frac{d P}{d t} = \frac{{{v}_{max} [ S ]}^{n}}{{K}^{n} + {[ S ]}^{n}} \qquad ( 16 b )$$

where Kⁿ=K₁·K₂· . . . ·Kₙ. When assessing the effect of an agonist A binding to a receptor R at one of the receptor's n catalytic centers we have to arrive at [S]=[AR], so according to the Michaelis-Menten model we will have the following equation:

$$[ A R ] = \frac{[ {R}_{0} ] [ A ]}{K + [ A ]} \qquad ( 17 a )$$

By combining both equations and by introducing a transducer ratio (defined as τ=[R₀]/K_{A}) we obtain a new equation that describes a contractual catalytic effect (following the original work by Black J W, Leff P (1983) Operational models of pharmacological agonism. Proc R Soc Lond B Biol Sci. 220(1219):141-62)

$$\frac{Z}{{Z}_{m}} = \frac{{{τ}^{n} [ A ]}^{n}}{{( {K}_{A} + [ A ] )}^{n} + {{τ}^{n} [ A ]}^{n}} \qquad ( 17 b )$$

which is a generalization of equations (16a) and (16b).

A transition effect between the resting and the hyperemic state is not only limited to regulation of an active coronary vasomotor tone. As a matter of fact, observed and investigated changes in a coronary circulation resistance are only a certain type of a symptom of the influence that is created by different cardiac tropism forms (including chronotropism, inotropism, lusitropism and many others).

In a preferred embodiment, a transition from the baseline to the hyperemic state is regarded as net cardiac tropism effects of purinergic receptors agonists binding and modeled by cooperative binding relation in the form of:

$$\frac{Z - {Z}_{min}}{{Z}_{max} - {Z}_{min}} = \frac{{X}^{n}}{{K}^{n} + {X}^{n}} \qquad ( 18 )$$

wherein: X is an arbitrary measurement reflecting changes in a ligand concentration, and K denotes an apparent dissociation constant. This equation represents one of the approaches to the PK/PD model.

In another embodiment of the invention, the net cardiac trophism effect is modeled by Black-Leff operational model of pharmacological agonism (following Black J W, Leff P (1983) Operational models of pharmacological agonism, Proc R Soc Lond B Biol Sci, 220(1219), p. 141-62, Leff P, Martin G R, Morse J M (1985) Application of the operational model of agonism to establish conditions when functional antagonism may be used to estimate agonist dissociation constants, Br J Pharmacol, 85(3), p. 655-63) and has the following form:

$$\frac{Z - {Z}_{min}}{{Z}_{max} - {Z}_{min}} = \frac{{τ}^{n} {X}^{n}}{{( K + X )}^{n} + {τ}^{n} {X}^{n}} \qquad ( 19 )$$

where: X and K—again—are notional values of concentration changes and a dissociation constant, while T is a ratio that defines the efficacy for an agonist (see Jambhekar S S, Breen P J (2012) Basic Pharmacokinetics, Pharmaceutical Press, Frigyesi A, Hdssjer O (2006) Estimating the parameters of the operational model of pharmacological agonism, Stat Med, 25(17), p. 2932-45).

All embodiments of the invention were tested. The method according to the invention was validated based on medical experiment results from a multi-center, non-randomized clinical trial. This study was planned to be performed on 60 participants. Finally, 67 patients were screened and 48 patients were enrolled. Gathered patients' demographic and health data that affect circulation hemodynamic in a body is presented in Table 1 (parameters are expressed as mean±standard deviation). For this purpose, specifically data that affect a pressure pulse propagation in patients' bodies.

TABLE 1
Baseline characteristics of the clinical experiment population

| Parameter | Female | Male | All cases |
|---|---|---|---|
| Number of cases | 30 (54.5%) | 25 (45.5%) | 55 (100%) |
| Myocardial ischemia | 8 (26.7%) | 17 (68.0%) | 25 (45.5%) |
| [among the total | | | |
| number of cases] | | | |
| Age [years] | 68.57 ± 8.49 | 67.12 ± 8.96 | 67.91 ± 8.65 |
| Height [cm] | 161.53 ± 5.67 | 174.24 ± 7.52 | 167.31 ± 9.12 |
| Weight [kg] | 72.00 ± 11.33 | 88.52 ± 14.09 | 79.51 ± 15.03 |
| HR [1/min] | 68.30 ± 5.06 | 68.72 ± 6.02 | 68.49 ± 5.47 |
| SYS [mmHg] | 131.83 ± 11.03 | 136.72 ± 13.46 | 134.05 ± 12.32 |
| DIA [mmHg] | 79.47 ± 9.93 | 82.56 ± 8.09 | 80.87 ± 9.19 |
| HCT [ml/100 ml] | 40.43 ± 3.12 | 42.20 ± 4.45 | 41.24 ± 3.85 |
| RBC [10³/μl] | 4.41 ± 0.42 | 4.58 ± 0.60 | 4.49 ± 0.51 |
| HGB [g/dl] | 13.49 ± 1.10 | 14.24 ± 1.69 | 13.83 ± 1.44 |
| PLT [10³/μl] | 241.17 ± 59.69 | 201.56 ± 50.95 | 223.16 ± 58.85 |
| BBLOCK | 21 (70.0%) | 17 (68.0%) | 38 (69.1%) |
| ACE | 7 (23.3%) | 12 (48%) | 19 (34.5%) |
| AARR | 22 (73.3%) | 17 (68%) | 39 (70.9%) |

HR—heart rate;
SYS and DIA—systolic and diastolic pressure, respectively;
HCT—hematocrit;
RBC, HGB, PLT—red blood cell, hemoglobin and platelets count, respectively;
BBLOCK—a beta-adrenergic blocking agent;
ACE—an angiotensin-converting-enzyme inhibitor;
AARR—an antiarrhythmic agent

Continuous non-invasive blood pressure measurements (CNBP) were performed on the radial artery with a sampling rate of 100 Hz were performed for each enrolled patient. In addition, an independent non-invasive measurement was performed in the brachial artery using the oscillometric method. Since the patients underwent invasive diagnostics, the results of intra-aortic catheter pressure measurements were used as reference data. The pressure signals from the intra-aortic catheter pressure measurements were sampled at a rate of 200 Hz.

Each patient enrolled to the clinical trial was diagnosed with a coronary heart disease, however the trials itself had to confirm a CHD by a functional assessment. This is presented in Table 2.

TABLE 2
In-vivo functional assessment of a coronary stenosis

| Group | | | | | | |
|---|---|---|---|---|---|---|
| of patients | Mean | Std. deviation | Min | Max | Skewness | Kurtosis |
| female | 0.79 | 0.07 | 0.68 | 0.91 | 0.29 | −0.98 |
| male | 0.76 | 0.10 | 0.54 | 0.94 | −0.21 | −0.38 |
| total | 0.77 | 0.09 | 0.54 | 0.94 | −0.27 | 0.06 |

Such a specific choice of a validation group—only people diagnosed with a coronary heart disease—reflected the target application focus of the invention.

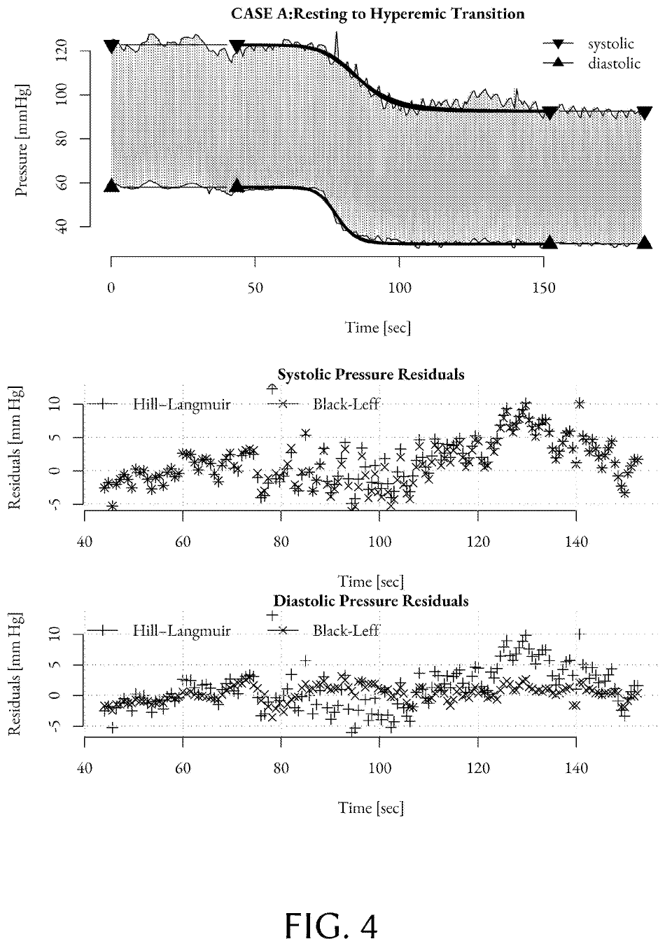

FIG. 4. is presenting results of an embodiment of method according to the invention incorporating a model based on Hill-Langmuir or Black-Leff equations. Both types of equations produce similar results and can be used interchangeably. In the background, there is a recording of 3 minutes of a left coronary artery catherization and prediction of systolic (triangle downward) and diastolic (triangle upward) pressure changes plotted with a solid line. In the resting state, a mean systolic pressure was at SYS=122.7±3.0 mmHg (which gave a coefficient of variation cv=2.4%), and a diastolic pressure was at DIA=57.9±1.51 mmHg (while cv=2.6%). Exactly at 43.7 s., Adenosine was administered and at 108.44 s. stable hyperemia was reached. Afterwards, the pressures decreased—SYS=92.5±1.5 mmHg (cv=1.6%) and DIA=32.1±0.6 mmHg (cv=1.8%). Basically, regardless of the used model (8) or (9), the predicted results were following the same pattern as it is shown by SYS and DIA pressure lines (which are coinciding). A mean residuum in a systolic values area for the model (8) was just 1.6 mmHg and did not exceed the range of −5.2-12.2 mmHg and for a diastolic pressure it was 0.4 in the range of −3.7-3.3 mmHg. Similarly for the model (9), the mean residuum for a systolic pressure was 1.3 mmHg for the range of range −6.0-13.1 mmHg and for a diastolic pressure 0.3 in the range of −3.6-3.2 mmHg. It should also be noted that CASE A presented at FIG. 4 expressed a reaction for an Adenosine receptors stimulation in the classic sigmoidal form. Statistically, this form is observed in around 57% of cases in clinical conditions—so a little more than for every second patient. A totally different reaction pattern is observed for around 39% of patients (i.e., for two in five patients that are put into the hyperemic state). For those patients, the reaction pattern is of humped type and therefore sigmoid with superimposed bumps of variation (see Johnson N P (2015) Repeatability of Fractional Flow Reserve Despite Variations in Systemic and Coronary Hemodynamics, JACC Cardiovasc Interv, 8(8), p. 1018-1027).

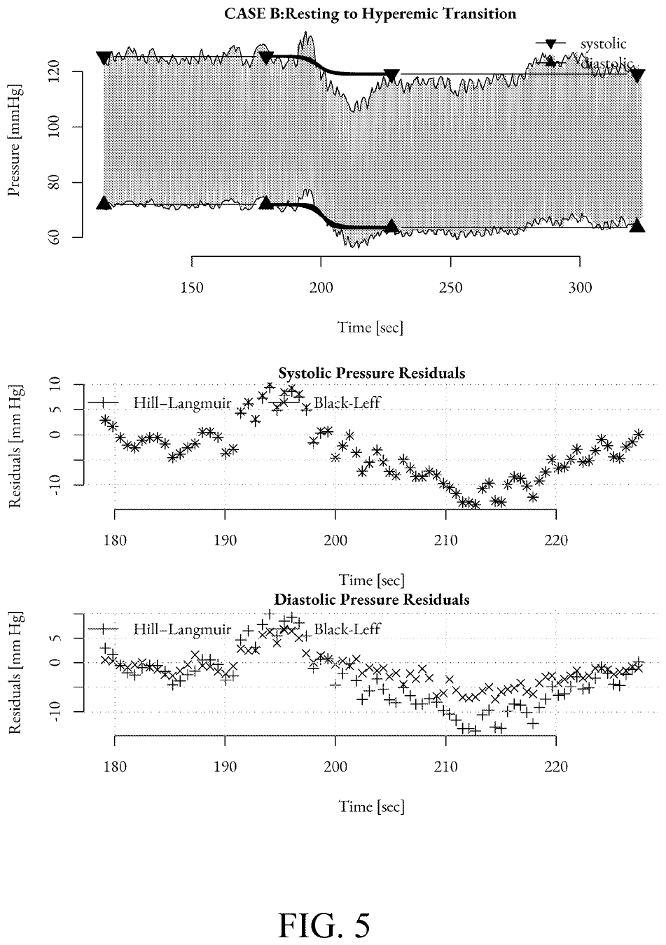

FIG. 5 shows a second pattern of reaction on Adenosine. On the resting state side, there is pressure SYS=125.5±2.1 mmHg (cv=1.7%) and DIA=71.9±1.2 mmHg (cv=1.7%). In fact, in the transitional phase much higher residuum values can be clearly seen and the used model did not have major impact on them. For the first model (equation 18) for a systolic pressure of −3.7 (−13.9 . . . 9.5) mmHg and for a diastolic pressure of −1.6 (−7.4 . . . 5.8) mmHg. For the second model (equation 19) there are −3.6 (−13.9 . . . 10.0) mmHg and −1.6 (−7.5 . . . 6.7) mmHg, respectively. Residuum values for a residuum of a humped type are higher in the transient phase, but that does not negatively affect the prediction quality of both models for the state of maximal stable hyperemia.

A method comprising a model formulated above using (18) and (19) gives possibilities for an effective mapping, with the use of the results of in vivo measurements made during patient's resting state (for example, a non-invasively measured central arterial pressure using the photoplethysmography method), from the resting state to the expected state under hyperemic conditions.

In a preferred embodiment of the present invention, a regression model providing the initiating values for internal empirical parameters of a three-component model (3CM) can be used (following Heijmans R D H, Magnus J R (1986) Consistent maximum-likelihood estimation with dependent observations. The general (non-normal) case and the normal case, J Econ, 32, p. 253-285, Escobar J (2012) Time-varying parameter estimation under stochastic perturbations using LSM, J Math Control Inform, 29(2), p. 235-258, Banks H T, Hu S, Thompson W C (2014) Modeling and inverse problems in the presence of uncertainty, CPC Press, Boca Raton, Mao Z (2020) Model validation and uncertainty quantification, Springer International Publishing). This model was used to process data from the trial.

As discussed above, clinical experiment population data were used for the parametric identification. They were used to estimate values of the empirical parameters of the 3CM. A one-time accomplishment of the parametrical identification gives a model that can be used for patient-specific calculations. This is specifically preferable in a routine usage when there is a need to give fast and precise starting values for specific case calculations.

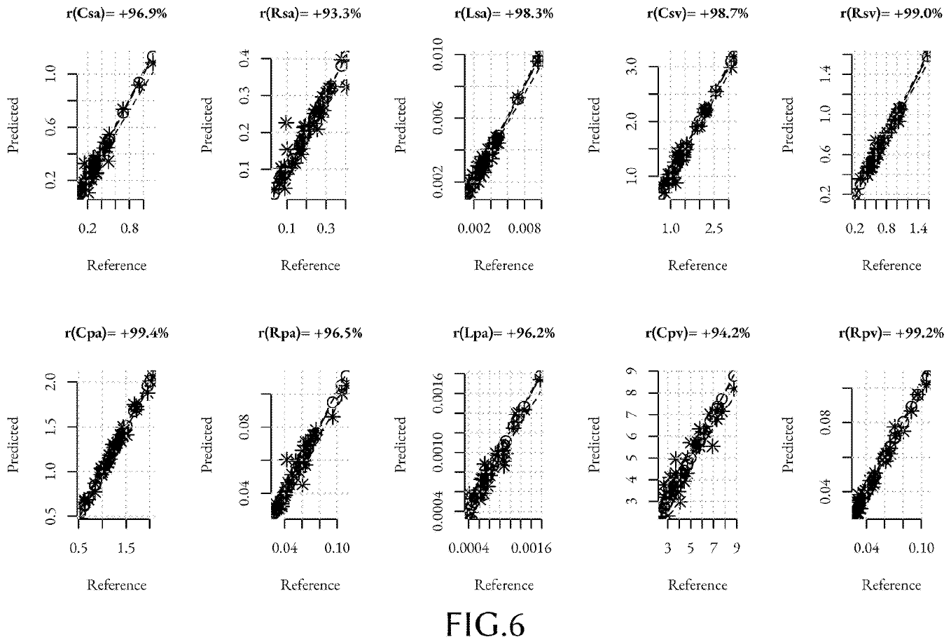

FIG. 6 shows the prediction results for selected empirical parameters of the 3CM wherein the 3CM had a structure as depicted in FIG. 3. In each of the graphs, the horizontal axis presents the values of the model parameters obtained as the result of calibration (parametric identification) relative to the values obtained with the use of invasive measurements (made in vivo for the state of hyperemia). The circle symbols mark the exact locations of values obtained using in vivo measurements. The calibration was performed separately for each patient participating in clinical trials, within the established window of five heart cycles. When assessing the quality of reproduction of the results of the invasive measurements, the L2 norm of pressure differences was calculated (in vivo vs. in silico). The value of the L2 norm was 2.3 mmHg for all cases and cycles (at the level of 95% confidence the interval was 2.1 . . . 2.5). The calibration resulted in the values of the empirical parameters that were, with statistically high probability, representative for the state of hyperemia for a specific patient.

Subsequently, for each of the parameters, a regression model was built using the demographic and general medical data. The results of this model are plotted on the vertical axis at FIG. 6 where the star symbols mark the values of a given empirical parameter obtained using the respective demographic and general medical data. Additionally, each graph contains a simple regression made with a solid line and a representation of a range that has the confidence level of 95% made with a dashed line. FIG. 6 presents only ten selected empirical parameters, out of several dozens that are included in the 3CM. The same type of markings is used in FIG. 3 and FIG. 6 for the same data. It is not difficult to notice that the results of the parametric identification for the 3CM using demographic and medical data and continues non-invasive measurements results have a very high correlation with the results obtained from the calibration made using invasive data (the value of the correlation is given as a percentage in the title of each plot). Adaptation of the method according to the invention to specific patients and their variability, described e.g., with the patient's demographic and medical data, is also clearly visible in FIG. 6. The use of the regression model is entirely optional for the invention and does not allow to obtain more precise results. It is noted that the regression model implementing statistical invasive data is able to provide much better starting values for the method of the invention. This yields several times faster conclusion of parametric identification of the model and, as the result, allows for arriving at patient-specific parameters calculated by the model much faster.

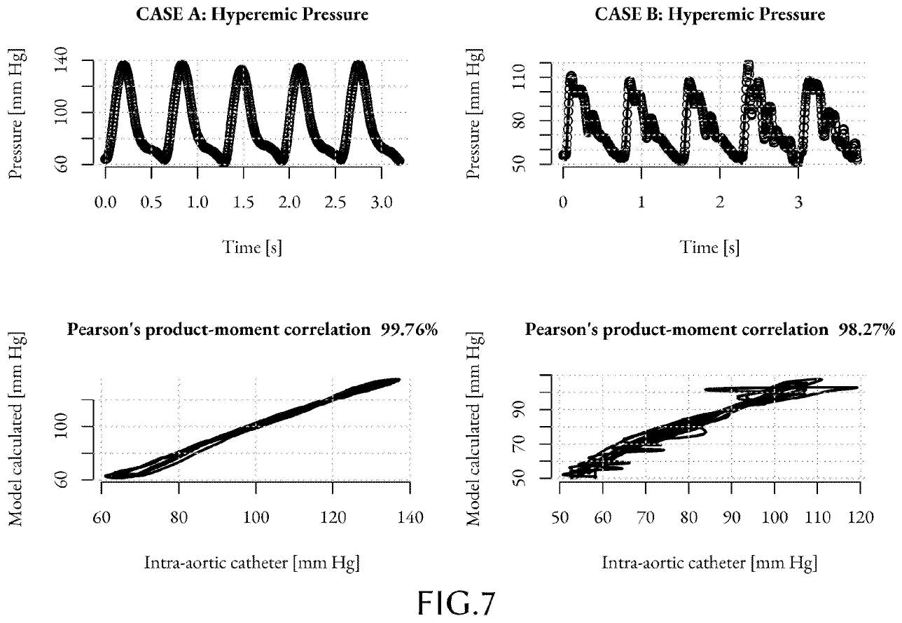

FIG. 7 presents selected results obtained with an embodiment of the method of the invention. The results are presented with the use of two extreme boundary cases. This selection allows for comparison. Both selected cases concerning the state of hyperemia: CASE A (left side) and CASE B (right side). The first case presented the most typical and the most common form of reaction of the systemic circulation to the administration of Adenosine, e.g., known from textbooks. The second (CASE B) was extremely unusual and peculiar. It should be noted that this is the only one of this kind in the pool selected for the clinical trials. This case was deliberately selected for comparison to benchmark the invention in the most harsh environment. Despite this, for both cases a very good agreement was reached between the results obtained using the method of the invention and the results obtained using the results obtained from invasive measurements. The agreement was assessed by calculating a Perason's correlation coefficient which reached the value of 99% (CASE A) and 98% (CASE B). The plot of the variability presented as a function of time of the calculated pressures (with a solid line in the graph) and the invasively measured pressures (as points in the form of a circle) also showed an excellent agreement. The fourth cycle for the CASE B a greater deviation in the maximum range (about 10 mmHg) was observed. This deviation was expected due to the extreme character of CASE B.

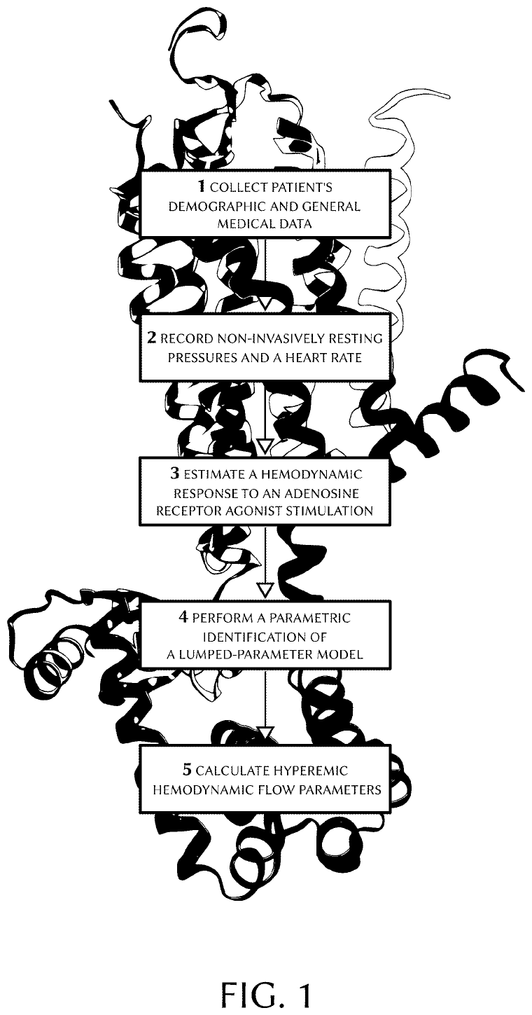

The method of the invention in one embodiment comprises five steps showed on FIG. 1. In the first step, the patient's demographic and general medical data are collected. As described earlier, this step is not needed for the method, but it may be beneficial for pace of calculations. While they are only used in step 4 of the method, which is related to the parametric identification of the model, their importance is relevant for the efficiency of the method. First of all, data relating to gender, age, weight and/or height are collected (influence of which, in one embodiment, is estimated according to Smulyan H et al. (1998) Influence of body height on pulsatile arterial hemodynamic data. Journal of the American College of Cardiology. 31(5):1103-9, Christofaro D G D et al. (2017) Relationship between Resting Heart Rate, Blood Pressure and Pulse Pressure in Adolescents. Arquivos Brasileiros de Cardiologia. 108(5):405-410, Evans J M et al. (2017) Body Size Predicts Cardiac and Vascular Resistance Effects on Men's and Women's Blood Pressure. Front Physiol. 9; 8:561, Gallo C et al. (2021) Testing a Patient-Specific In-Silico Model to Noninvasively Estimate Central Blood Pressure. Cardiovascular Engineering and Technology. 12(2):144-157). Drug therapies can also influence pulse wave propagation. In particular, the drug therapies that involve beta-adrenergic blocking agents (BBLOCK), angiotensin-converting-enzyme inhibitor (ACE) and/or antiarrhythmic agent (AARR) are relevant (influence of which, in one embodiment, is estimated according to Harris W S, Schoenfeld C D, Weissler A M (1967) Effects of adrenergic receptor activation and blockade on the systolic preejection period, heart rate, and arterial pressure in man. Journal of Clinical Investigation. 46(11):1704-14, Morgan T O et al. (1974) A comparison of beta adrenergic blocking drugs in the treatment of hypertension. Postgraduate Medical Journal. 50(583):253-259, Nyberg G (1976) Effect of beta-adrenoreceptor blockers on heart rate and blood pressure in dynamic and isometric exercise. Drugs. 11 SUPPL 1:185-95, Fitzpatrick M A, Julius S (1985) Hemodynamic effects of angiotensin-converting enzyme inhibitors in essential hypertension: a review. Journal of Cardiovascular Pharmacology. 7 Suppl 1:S35-9, Ting C T et all (1993) Arterial hemodynamics in human hypertension. Effects of angiotensin converting enzyme inhibition. Hypertension. 22(6):839-46, Jobs A et al. (2019) Angiotensin-converting-enzyme inhibitors in hemodynamic congestion: a meta-analysis of early studies. Clinical Research in Cardiology. 108(11):1240-1248, Block P J, Winkle R A (1983) Hemodynamic effects of antiarrhythmic drugs. American Journal of Cardiology. 52(6):14C-23C, Weiner B (1991) Hemodynamic effects of antiarrhythmic drugs. Journal of Cardiovascular Nursing. 5(4):39-48). In some embodiments, drug therapies are included in the method, in other embodiments drug therapies comprise the drugs listed above. Other drugs may also affect pulse wave propagation in a patient's body and, as the result, other embodiments comprise other drug therapies. It should be noted that drug therapies include also different dosing regimes as well as any further medical effects of drug therapies. Each of the above-mentioned factors can be used individually or combined in any fashion to obtain better starting values (initiating) for the model identification process for a particular patient's case. Better starting values directly affect efficiency of the method according to the invention. It is contemplated that in certain embodiments only selected data are collected, e.g., gender, age or selected medications. The more data is used, the better starting values can be achieved.

In the second step of method of the invention, as shown in FIG. 1, a resting state patient's pressure waveform is acquired. This step is also not necessary for the invention but may be useful in parametric identification step. Alternatively or additionally, this step may be used to assume main hemodynamic parameters for the purpose of the third step. Considering the limitations and preferences of clinical practice in measuring continuous non-invasive blood pressure (CNBP), in a preferred embodiment of the present invention a pressure measurement is made in the radial artery. In one embodiment, the pressure measurement is done using the finger-cuff photoplethysmography and/or the applanation tonometry. Benefits of using said measurement techniques is that they are simple to use and the invention will work even for those techniques. The recording and further analysis of a continuous pressure waveform is performed within a window that comprises blocks of signals that covers one or more complete cycles of a patient's heart. Each cycle corresponds to the systolic-diastolic action of a heart. In the preferred embodiment of the present invention, a window with a width corresponding to the length of a respiratory cycle can be analyzed (following Rodriguez-Molinero A (2013) Normal respiratory rate and peripheral blood oxygen saturation in the elderly population. Journal of the American Geriatrics Society. 61(12):2238-2240, Park C, Lee B (2014) Real-time estimation of respiratory rate from a photoplethysmogram using an adaptive lattice notch filter. Biomedical Engineering Online. 17; 13:170, Scholkmann F, Wolf U (2019) The Pulse-Respiration Quotient: A Powerful but Untapped Parameter for Modern Studies About Human Physiology and Pathophysiology. Front Physiol. 9; 10:371). This embodiment is beneficial as it allows for a more precise assumption of the parameters.

In the third step of method of the invention, as shown in FIG. 1, main hemodynamic parameters of a patient for the hyperemic state are determined. This is done using the process of mapping during which the parameters that describe the resting state are transpose to the parameters that describe the hyperemic state. The mapping of parameters obtained using the results of non-invasive measurements made in the resting state is done using PK/PD-model with equations (18) and (19). In one embodiment, mapping can be done only for the main hemodynamic parameters that include a systolic (SYS) and a diastolic (DIA) pressure and a heart rate (HR). In another embodiment, further hemodynamic parameters can be used. In one embodiment, the PK/PD-model is not used directly for the empirical parameters of the 3CM (with the structure as shown on FIG. 3). In another embodiment, waveform is not used in the PK/PD-model and waveform of the hyperemic state is provided by the 3CM. This embodiment is much more time efficient in comparison to embodiments in which the waveform is calculated using the PK/PD-model.

In the fourth step, a parametric identification of a lumped parameter 3CM of hemodynamics is performed (FIG. 3). For this purpose, a regression model can be used. The regression model provides a stochastic quantitative description. The regression model can be used to provide starting values for the 3CM. It is noted that in one embodiment, no regression model is used in the method which requires additional time for calculations to be completed. This additional time is approaching several dozen of hours. The embodiments with the regression model arrive at the results faster and the parametric identification in that embodiments is a more numerically stable process. The parametric identification, in another embodiment, is made independently and in parallel with an Adenosine receptor stimulation. Refining of values of the empirical parameters using hyperemic hemodynamic parameters can be done using any suitable mathematical method.

In the fifth step, one or more flow parameters in the systemic, the pulmonary and/or the coronary circulation are calculated through the three-component model (3CM). Said flow parameters include flow rates and pressures in locations corresponding to the used structure of the 3CM. A particular structure of the 3CM has a minor importance for heart chambers flow parameters (in each case there are two atriums and ventricles), but for the blood circulation system (BCS) the differences can be important. The embodiment showed on FIG. 3 which has two compartments for the systemic circulation and two compartments for the pulmonary circulation of the BCS model provides flow rates and pressures pertaining to those compartments only (to be exact, for the artery and venous reservoir). In different embodiments, there is a n-compartment model for n higher than 2 is used. These embodiments provide a more detailed description at various levels of circulation systems. In a preferred embodiment of the invention, the coronary flow parameters are calculated for the left (LCA) and the right (RCA) arterial inlet.

Any calculation step or substep of the method according to the invention can be implemented using a computer or a computer program. In some specific embodiments, some or all calculations are done using a computer program stored on a computer or on any type of a memory device or both. In another embodiments, some or all calculations for the purpose of the method can be done remotely e.g., using a cloud-based infrastructure which can include the use of Internet or a local network, at a different place from a place where a patient is.

While all measurement methods and algorithms described here are intended to define the parameters of the invention and to provide tangible results to be compared with clinical trials, there are by no means limiting and are exemplary. The words like “including” or “comprising” are not limiting and, for example, when an element A includes another element B, the element A may include other element or elements in addition to the element B. The use of a singular or plural form is not limiting for the scope of the disclosure and, for example, a part of the description which is indicating that an element A contains an element B, this part also discloses that multiple elements B are contained in one element A and multiple elements A are contained in one element B as well as an embodiment where multiple elements A contain multiple elements B. Many other embodiments will be apparent and clear for those skilled in the art upon reviewing the content of the present disclosure. The scope of the invention, therefore, should be determined with reference to the appended claims along with the full scope of equivalents to which said claims are entitled to.

1. A method for assessment of a hemodynamic response to an Adenosine receptor agonist stimulation for a human patient, wherein the method comprises the steps of:
mapping of resting hemodynamic parameters of the human patient to hyperemic hemodynamic parameters of the human patient using a pharmacokinetic/pharmacodynamic model, wherein the pharmacokinetic/pharmacodynamic model is a non-linear model that includes Michaelis-Menten, Hill-Langmuir, Black-Leff, multi-pathway and/or multi-species blood-tissue exchange model,
wherein the resting hemodynamic parameters include a patient's systolic pressure, a patient's diastolic pressure and/or a patient's heart rate, and the resting hemodynamic parameters are measured non-invasively in the resting state or are assumed using a non-invasively recorded continuous patient's pressure waveform,
wherein the non-invasively recorder continuous patient's pressure waveform is of the human patient in the resting state, wherein the non-invasively recorded continuous patient's pressure waveform comprises a time window that includes at least one entire cycle of a heart of the human patient, and
performing a parametric identification of a lumped-parameter model of the human patient using the hyperemic hemodynamic parameters of the human patient to arrive at the lumped-parameter model of the human patient in the hyperemic state, wherein the lumped-parameter model describes at least in part hemodynamics of the human patient and is of a Windkessel type.

2. The method of claim 1, wherein the method further comprises calculating of flow parameters using the lumped-parameter model of the human patient in the hyperemic state, wherein the flow parameters include a pressure, a flow rate, a waveform and/or cardiac time intervals that include, but are not limited to, a heart cycle duration and an ejection time.

3. The method of any of claims 1-2, wherein the resting hemodynamic parameters of the human patient further include a non-invasively recorded continuous resting patient's pressure waveform.

4. The method of any of claims 1-3, wherein the parametric identification comprises the following steps:
estimating of values of empirical parameters of the lumped-parameter model of the human patient using demographic and health data that affect pressure pulse propagation in human patients' bodies, and
refining the values of the empirical parameters of the lumped-parameter model of the human patient using the hyperemic hemodynamic parameters of the human patient.

5. The method of claim 4, wherein the demographic and health data are gathered for patients that are diagnosed with a coronary heart disease.

6. The method of any of claims 4-5, wherein the demographic and health data include gender, age, body height, general fitness assessment and/or current medication, wherein the current medication includes, but is not limited to, a beta-adrenergic blocking agent, an angiotensin-converting-enzyme inhibitor and/or an antiarrhythmic agent.

7. The method of any of claims 1-6, wherein the Black-Leff model comprises the following equation:
$$\frac{Z}{{Z}_{m}} = \frac{{{τ}^{n} [ A ]}^{n}}{{( {K}_{A} + [ A ] )}^{n} + {{τ}^{n} [ A ]}^{n}}$$
wherein Z is a pharmacological effect; Zₘ is a maximum response; τ is a ratio defining the efficacy of an agonist; [A] is a concentration of an agonist; K_{A} is an agonist-receptor dissociation constant; and n is a factor determining the steepness of a curve.

8. The method of any of claims 1-7, wherein the Hill-Langmuir model comprises the following equation:
$$\frac{Z - {Z}_{min}}{{Z}_{max} - {Z}_{min}} = \frac{{X}^{n}}{{K}^{n} + {X}^{n}}$$
wherein Z is a pharmacological effect; Zₘₐₓ is a maximum response; Zₘᵢₙ is a minimum response; X is an arbitrary measurement reflecting changes in an agonist concentration; K is an apparent dissociation constant; and n is a factor determining the steepness of a curve.

9. The method of any of claims 1-8, wherein the Black-Leff model comprises the following equation:
$$\frac{Z - {Z}_{min}}{{Z}_{max} - {Z}_{min}} = \frac{{τ}^{n} {X}^{n}}{{( K + X )}^{n} + {τ}^{n} {X}^{n}}$$
wherein Z is a pharmacological effect; Zₘₐₓ is a maximum response; Zₘᵢₙ is a minimum response; X is an arbitrary measurement reflecting changes in an agonist concentration; K is an apparent dissociation constant; n is a factor determining the steepness of a curve; and τ is a ratio defining the efficacy of an agonist.

10. The method of any of claims 1-9, wherein the non-invasively recorded continuous patient's pressure waveform is recorded on the radial artery.

11. The method of any of claims 3-10, wherein the non-invasively recorded continuous resting patient's pressure waveform is recorded on the radial artery.

12. The method of any of claims 1-11, wherein the non-invasively recorded continuous patient's pressure waveform is recorded using methods selected from photoplethysmography and/or applanation tonometry.

13. The method of any of claims 3-12, wherein the non-invasively recorded continuous resting patient's pressure waveform is recorded using methods selected from photoplethysmography and/or applanation tonometry.

14. The method of any of claims 1-13, wherein the time window comprises at least one whole respiratory cycle.

15. The method of any of claims 1-14, wherein the lumped-parameter model of the human patient is a three-component model that comprises:
a blood circulatory system (BCS) component, wherein the BCS component comprises one, two or more CRL functional blocks,
a heart chamber pressure-volume (HPV) component, wherein the HPV component comprises one, two or more ERv functional blocks, and
a coronary blood flow (CBF) component, wherein the CBF component comprises one, two or more RCpRp functional blocks, and
wherein the CRL functional block comprises the following structure: 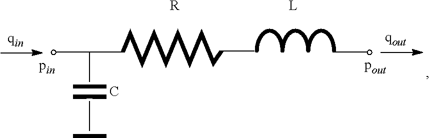
and
wherein the ERv functional block comprises the following structure: 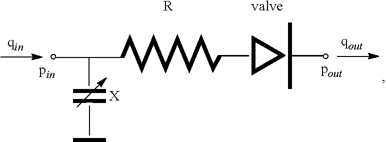
and
wherein the RCpRp functional block comprises the following structure: 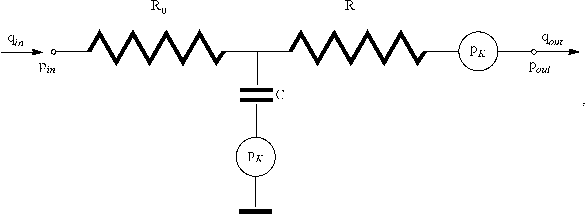
and
wherein pᵢₙ, qᵢₙ is a pressure and a flow rate at the inlet of a compartment; pₒᵤₜ, qₒᵤₜ is a pressure and a flow rate at the outlet of a compartment; R₀, R, L, C is a proximal and a distal resistance, an inertance and a compliance; valve is a heart valve modeling diode; p_{C}, p_{R} is a myocardium vessel interaction (MVI) pressure, wherein C is a compliant and R is resistive arteries; and X is E or MF, wherein E is the time-varying elastance concept and MF is the myocardial fiber stress and strain concept.

16. The method of claim 15, wherein the myocardium vessel interaction (MVI) includes calculating a coronary flow throttling pressure:
$${p}_{R , C} = {k}_{R , C} ( CEP + SIP ) = {k}_{R , C} ( {μ}_{1} p ( t ) + {μ}_{2} {E}_{n} ( t ) ) ,$$
wherein p is an interstitial, intracavity pressure; k_{R,C} is a resistive and compliant part of a coronary tree coefficient; μ₁ is a cavity-induced extracellular pressure coefficient; μ₂ is a shortening-induced intracellular pressure; and Eₙ is normal elastance.

17. The method of any of claims 15-16, wherein each said RCpRp functional block satisfies the equation:
$${q}_{in} = C \frac{d}{dt} ( {p}_{in} - {R}_{0} {q}_{i n} - {p}_{C} ) + \frac{〈 {p}_{i n} - {R}_{0} {q}_{i n} - {p}_{R} - {p}_{zf} 〉}{R} + {q}_{o u t} ,$$
wherein p_{zf} is zero flow pressure in coronary flow, and/or
wherein each said CRL functional block satisfies the equation:
$$\{ \begin{matrix}{q}_{i n} = C \frac{d {p}_{i n}}{dt} + {q}_{out} \\ {p}_{i n} = R {q}_{out} + L \frac{d {q}_{out}}{d t} + {p}_{out}\end{matrix} ,$$
wherein C, R and L are compliance, resistance and inertance, respectively.

18. The method of claim 17, wherein the zero flow pressure is equal 14.8±7 mmHg for patients with a stable angina pectoris or 22.5±9.1 mmHg for patients with a non-Q-wave myocardial infraction or 37.1±1.9 mmHg for patients with a Q-wave myocardial infraction.

19. The method of any of claims 15-18, wherein the lumped-parameter model of the human patient comprises a systemic and pulmonary circulation model, wherein the systemic circulation comprises a left heart circuit, and the pulmonary circulation comprises a right heart circuit, and wherein each circuit has a form of at least two said CRL functional blocks serially connected.

20. The method of any of claims 15-19, wherein the lumped-parameter model of the human patient comprises the following structure: 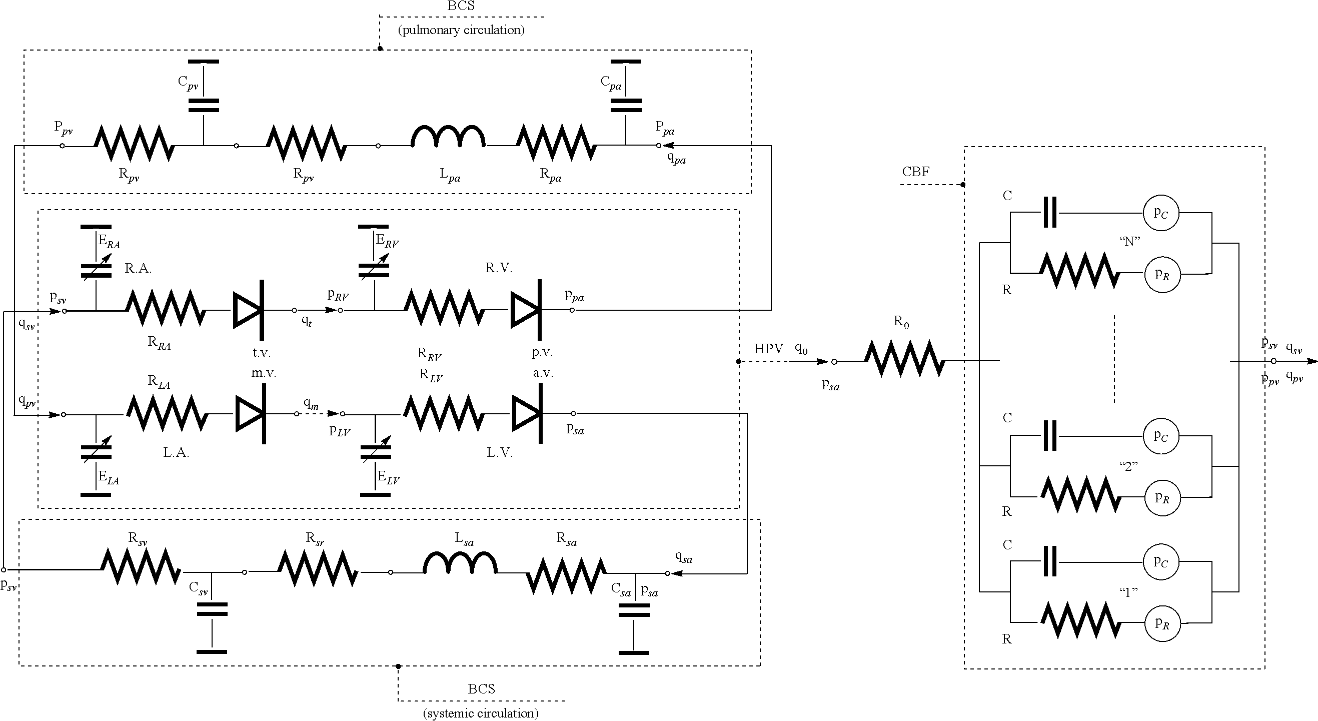
wherein p is pressure; q is flow rate; pa is arteries; pr is reservoir; pv is veins; sa is aorta; sp is proximal arteries; sd is distal arteries; sr is reservoir; sv is veins; L is inertance; R is resistance; C is compliance; X is E or MF, wherein E is the time-varying elastance concept and MF is the myocardial fiber stress and strain concept; R.A., R.V. is right atrium and ventricle; L.A., L.V. is left atrium and ventricle; t.v. is tricuspid (atrio-ventricular) valve; p.v. is pulmonary (ventricular) valve; m.v. is mitral (atrio-ventricular) valve; and a.v. is aortic (ventricular) valve.

21. A computer-readable [storage] medium comprising instructions which, when executed by a computer, cause the computer to carry the steps of a method defined in any of claims 1-20.

22. A system for assessment of a hemodynamic response to an Adenosine receptor agonist stimulation for a human patient, wherein the system comprises:
a measuring means for non-invasively measuring resting hemodynamic parameters of the human patient, wherein the resting hemodynamic parameters include a patient's systolic pressure, a patient's diastolic pressure and/or a patient's heart rate, and
a computer system adapted to perform the steps of a method defined in any of claims 1-20.

23. The system for assessment of a hemodynamic response to an Adenosine receptor agonist stimulation for a human patient of claim 22, wherein the computer system further comprises a registering means for non-invasive continuous registration of a resting pressure waveform for the human patient.

24. The system for assessment of a hemodynamic response to an Adenosine receptor agonist stimulation for a human patient of any of claims 22-23, wherein the computer system comprises:
at least one computer adapted to perform mapping of resting hemodynamic parameters of the human patient to hyperemic hemodynamic parameters of the human patient using a pharmacokinetic/pharmacodynamic model, and
at least one computer adapted to perform a parametric identification of a lumped-parameter model of the human patient using the hyperemic hemodynamic parameters of the human patient to arrive at the lumped-parameter model of the human patient in the hyperemic state, and
wherein said at least one computer adapted to perform mapping of resting hemodynamic parameters of the human patient to hyperemic hemodynamic parameters of the human patient using a pharmacokinetic/pharmacodynamic model is configured to communicate directly or indirectly with said at least one computer adapted to perform a parametric identification of a lumped-parameter model of the human patient using the hyperemic hemodynamic parameters of the human patient to arrive at the lumped-parameter model of the human patient in the hyperemic state.
